(19) United States
(12) Patent Application Publication
(10) Pub. No.: US 2022/0249438 A1
(43) Pub. Date: Aug. 11, 2022

(54) CARBOCYANINE COMPOUNDS FOR TARGETING MITOCHONDRIA AND ERADICATING CANCER STEM CELLS

(71) Applicant: LUNELLA BIOTECH, INC., Ottawa, ON (CA)

(72) Inventors: Michael P. LISANTI, Manchester (GB); Federica SOTGIA, Manchester (GB); Camillo SARGIACOMO, Salford (GB)

(21) Appl. No.: 17/622,512

(22) PCT Filed: Jun. 26, 2020
(86) PCT No.: PCT/US2020/039744

§ 371 Date: Dec. 23, 2021

Related U.S. Application Data
(60) Provisional application No. 62/866,875, filed on Jun. 26, 2019.

(51) Int. Cl. A61K 31/404; A61K 31/375; A61K 31/7004; A61K 31/65; A61K 31/167; A61K 31/4745; A61P 35/00

(52) U.S. Cl. CPC A61K 31/404; A61K 31/375; A61K 31/7004; A61P 35/00; A61K 31/167; A61K 31/4745; A61K 31/65

(57) ABSTRACT

Certain carbocyanine compounds target mitochondria and may be used for eradicating cancer stem cells (CSCs). For example, MitoTracker Deep Red (MTDR) is a non-toxic, carbocyanine-based, far-red, fluorescent probe that is routinely used to chemically mark and visualize mitochondria in living cells. MTDR inhibits 3D mammosphere formation in MCF7 cells, MDA-MB-231 cells, and MDA-MB-468 cells, with an 1C-50 between 50 to 100 nM. Also, MTDR exhibited near complete inhibition of mitochondrial oxygen consumption rates and ATP production, in all three breast cancer cell lines tested, at a level of 500 nM. Nano-molar concentrations of MTDR can be used to specifically target and eradicate CSCs, by selectively interfering with mitochondrial metabolism. Other carbocyanine compounds having anti-CSC activity are described.

### RELATED APPLICATIONS

This application claims the benefit of U.S. provisional patent application 62/866,875, filed Jun. 26, 2019, and incorporated herein by reference in its entirety.

### FIELD

The present disclosure relates to therapeutic carbocyanine compounds and uses of such compounds for inhibiting mitochondrial function, and targeting and eradicating cancer stem cells (CSCs), and treating cancer.

### BACKGROUND

Researchers have struggled to develop new anti-cancer treatments. Conventional cancer therapies (e.g. irradiation, alkylating agents such as cyclophosphamide, and anti-metabolites such as 5-Fluorouracil) have attempted to selectively detect and eradicate fast-growing cancer cells by interfering with cellular mechanisms involved in cell growth and DNA replication. Other cancer therapies have used immunotherapies that selectively bind mutant tumor antigens on fast-growing cancer cells (e.g., monoclonal antibodies). Unfortunately, tumors often recur following these therapies at the same or different site(s), indicating that not all cancer cells have been eradicated. Relapse may be due to insufficient chemotherapeutic dosage and/or emergence of cancer clones resistant to therapy. Hence, novel cancer treatment strategies are needed.

Advances in mutational analysis have allowed in-depth study of the genetic mutations that occur during cancer development. Despite having knowledge of the genomic landscape, modem oncology has had difficulty with identifying primary driver mutations across cancer subtypes. The harsh reality appears to be that each patient's tumor is unique, and a single tumor may contain multiple divergent clone cells. What is needed, then, is a new approach that emphasizes commonalities between different cancer types. Targeting the metabolic differences between tumor and normal cells holds promise as a novel cancer treatment strategy. An analysis of transcriptional profiling data from human breast cancer samples revealed more than 95 elevated mRNA transcripts associated with mitochondrial biogenesis and/or mitochondrial translation. Additionally, more than 35 of the 95 upregulated mRNAs encode mitochondrial ribosomal proteins (MRPs). Proteomic analysis of human breast cancer stem cells likewise revealed the significant overexpression of several mitoribosomal proteins as well as other proteins associated with mitochondrial biogenesis.

Mitochondria are extremely dynamic organelles in constant division, elongation and connection to each other to form tubular networks or fragmented granules in order to satisfy the requirements of the cell and adapt to the cellular microenvironment, The balance of mitochondrial fusion and fission dictates the morphology, abundance, function and spatial distribution of mitochondria, therefore influencing a plethora of mitochondrial-dependent vital biological processes such as ATP production, mitophagy, apoptosis, and calcium homeostasis. In turn, mitochondrial dynamics can be regulated by mitochondrial metabolism, respiration and oxidative stress. Thus, it is not surprising that an imbalance of fission and fusion activities has a negative impact on several pathological conditions, including cancer. Cancer cells often exhibit fragmented mitochondria, and enhanced fission or reduced fusion is often associated with cancer, although a comprehensive mechanistic understanding on how mitochondrial dynamics affects tumorigenesis is still needed.

An intact and enhanced metabolic function is necessary to support the elevated bioenergetic and biosynthetic demands of cancer cells, particularly as they move toward tumor growth and metastatic dissemination. Not surprisingly, mitochondria-dependent metabolic pathways provide an essential biochemical platform for cancer cells, by extracting energy from several fuels sources.

Cancer stem-like cells (CSCs) are a relatively small sub-population of tumor cells that share characteristic features with normal adult stem cells and embryonic stem cells. As such, CSCs are thought to be a ‘primary biological cause’ for tumor regeneration and systemic organismal spread, resulting in the clinical features of tumor recurrence and distant metastasis, ultimately driving treatment failure and premature death in cancer patients undergoing chemo- and radio-therapy. Evidence indicates that CSCs also function in tumor initiation, as isolated CSCs experimentally behave as tumor-initiating cells (TICS) in pre-clinical animal models. As approximately 90% of all cancer patients die pre-maturely from metastatic disease world-wide, there is a great urgency and unmet clinical need, to develop novel therapies for effectively targeting and eradicating CSCs. Most conventional therapies do not target CSCs and often increase the frequency of CSCs, in the primary tumor and at distant sites.

Recently, energetic metabolism and mitochondrial function have been linked to certain dynamics involved in the maintenance and propagation of CSCs, which are a distinguished cell sub-population within the tumor mass involved in tumor initiation, metastatic spread and resistance to anti-cancer therapies. For instance, CSCs show a peculiar and unique increase in mitochondrial mass, as well as enhanced mitochondrial biogenesis and higher activation of mitochondrial protein translation. These behaviors suggest a strict reliance on mitochondrial function. Consistent with these observations, an elevated mitochondrial metabolic function and OXPHOS have been detected in CSCs across multiple tumor types.

One emerging strategy for eliminating CSCs exploits cellular metabolism. CSCs are among the most energetic cancer cells. Under this approach, a metabolic inhibitor is used to induce ATP depletion and starve CSCs to death. So far, the inventors have identified numerous FDA-approved drugs with off-target mitochondrial side effects that have anti-CSC properties and induce ATP depletion, including, for example, the antibiotic Doxycycline, which functions as a mitochondrial protein translation inhibitor. Doxycycline, a long-acting Tetracycline analogue, is currently used for treating diverse forms of infections, such as acne, acne rosacea, and malaria prevention, among others. In a recent Phase II clinical study, pre-operative oral Doxycycline (200 mg/day for 14 days) reduced the CSC burden in early breast cancer patients between 17.65% and 66.67%, with a near 90% positive response rate.

However, certain limitations restrain the use of sole anti-mitochondria. agents in cancer therapy, as adaptive mechanisms can be adopted in the tumor mass to overcome the lack of mitochondrial function. These adaptive mechanisms include, for example, the ability of CSCs to shift from oxidative metabolism to alternate energetic pathways, in a multi-directional process of metabolic plasticity driven by both intrinsic and extrinsic factors within the tumor cells, as well as in the surrounding niche. Notably, in CSCs the manipulation of such metabolic flexibility can turn as advantageous in a therapeutic perspective. What is needed, then are therapeutic approaches that either prevent these metabolic shifts, or otherwise take advantage of the shift to inhibit cancer cell proliferation.

It is therefore an object of this disclose to identify mitochondrial metabolic inhibitors that selectively target and eradicate CSCs. It is another object of this disclosure to identify new anti-cancer therapeutic approaches involving new pharmaceutical compounds that metabolically starve CSCs by targeting mitochondria and driving ATP depletion.

### SUMMARY

The present approach describes carbocyanine compounds, and in particular heptamethine cyanine compounds, that inhibit cellular metabolism and eradicate cancer cells and CSCs. As used herein, the term “carbocyanine” refers to a cyanine compound in which two heterocycline rings, normally quinoline groups, are joined by a polymethine bridge.

In some embodiments of the present approach, MitoTracker Deep Red (MTDR) is repurposed as a therapeutic compound for targeting mitochondrial metabolism in CSCs. (“MitoTracker” is a registered trademark of Molecular Probes, Inc.) MTDR, also known as 1-{4-[(chloromethyl)phenyl]methyl}-3,3-dimethyl-2-[5-(1,3,3-trimethyl-1,3-dihydro-2H-indol-2-ylidene)penta-1,3-dien-1-yl]-3H-indolium chloride, is a relatively non-toxic, carbocyanine-based, far-red, fluorescent probe that is routinely used to chemically mark and visualize mitochondria in living cells. MTDR can also be used as a marker to purify drug-resistant CSC activity by flow-cytometry, which was validated by functional assays, including pre-clinical animal models that documented higher tumor-initiating activity in vivo. As described herein, MTDR has potent mitochondrial metabolism inhibition properties, and is highly selective towards metabolically-active cancer cells, and in particular, CSCs.

In some embodiments, structural analogs of MTDR are used as therapeutic compounds for targeting mitochondrial metabolism in CSCs. The MTDR structural analogs having mitochondrial metabolism inhibition properties are described more fully below.

In addition to MTDR and its analogs, other near-infrared (NIR) cyanine compounds such as HITC and DDI, accumulate in MCF7 cells and inhibit CSC anchorage-independent growth. For example, results discussed below demonstrate that HITC effectively blocks CSCs growth in a mitochondrial-dependent manner, and induces glycolysis starting at 500 nM. In contrast, DDI does not produce any noticeable metabolic effects, but nonetheless inhibits CSC growth in the nanomolar range in MCF7 cells. Furthermore, at the nanomolar concentrations tested, IR-780 showed no effect on CSC growth, and was not internalized by tumor cells. Thus, under the present approach, NIR cyanine compounds may be screened for anti-mitochondrial effects and CSC propagation inhibition effects, to identify new mitochondrial metabolism inhibitors and anti-cancer therapeutic compounds.

The present approach also contemplates other heptamethine cyanine compounds, also referred to as “Cy5” cyanine analogs. Numerous Cy5 analogs having with different reactive groups were analyzed MCF7 CSC growth inhibition. The MCF7 cells internalized each of the tested Cy5 analogs after five days of treatment. The Cy5 analogs identified as Cy5-Alkyne and Cy5-Azide blocked mammosphere growth and also targeted the energized mitochondria in cancer cells within a nanomolar range. Thus, under the present approach, Cy5 analogs may be screened for anti-mitochondrial effects and CSC propagation inhibition effects, to identify new mitochondrial metabolism inhibitors and anti-cancer therapeutic compounds.

As set forth herein, the compounds of the present approach exploit the energetic state of malignant cancer cells, and can selectively target the CSCs. The in vitro findings described below show that carbocyanine-induced mitochondrial cytotoxicity of the compounds of the present approach may be used to prevent CSC-driven metastatic growth, and may be used as a therapeutic approach for the preventive treatment against cancer relapse (metastasis and/or recurrence), including before and after chemotherapy or radiation therapy.

In some embodiments, the carbocyanine compound induces a metabolic shift in CSCs, from an oxidative state to a glycolytic state. After this metabolic shift, CSC dependency on glycolysis may be used to eradicate the residual glycolytic CSC population through additional metabolic stressors. A carbocyanine compound may be combined with a second metabolic inhibitor to provide a “two-hit” therapeutic strategy. The selected second metabolic inhibitor may be chosen from natural and synthetic compounds, some of which are FDA-approved, known to behave as glycolysis inhibitors (e.g., Vitamin C, 2-Deoxy-Glucose or 2DG) or OXPHOS inhibitors (e.g., Doxycycline, Niclosamide, Berberine Chloride) inhibitors. Embodiments of the “two-hit” therapeutic strategy effectively decreased CSC propagation, at concentrations of the carbocyanine compound toxic only to cancer cells, but not to normal cells.

Under the present approach, pharmaceutical compositions may include a pharmaceutically effective amount of a carbocyanine compound, such as MTDR, a MTDR analog, or a Cy5 analog, which includes pharmaceutically acceptable salts thereof, and a pharmaceutically acceptable carrier, diluent, or excipient therefor. Some embodiments of the pharmaceutical composition may also include a pharmaceutically effective amount of a second metabolic inhibitor compound, such as a glycolysis inhibitor or an OXPHOS inhibitor. The second metabolic inhibitor compound may, in some embodiments, be in a separate pharmaceutically acceptable carrier. Compounds according to the present approach may be used as anti-cancer therapeutics. Pharmaceutically-effective amounts of compounds according to the present approach may be administered to a subject according to means known in the art. The carbocyanine compound may be co-administered with a second metabolic inhibitor compound in some embodiments. Alternatively, carbocyanine compound may be administered prior to, and optionally before and with, a second metabolic inhibitor. Compounds of the present approach may be administered to treat a cancer, to eradicate CSCs, to prevent or reduce the likelihood of tumor recurrence, and to prevent or reduce the likelihood of metastasis. In some embodiments, a pharmaceutically effective amount of a carbocyanine compound may be administered to cause a cancer to shift to a glycolytic state. in some embodiments, a pharmaceutically effective amount of a carbocyanine compound may be administered to increase the effectiveness of a chemotherapy. In some embodiments, a pharmaceutically effective amount of a carbocyanine compound may be administered to treat, prevent, and/or reduce the likelihood of at least one of tumor recurrence and metastasis, drug resistance, and radiotherapy resistance.

These and other embodiments will be apparent to the person having an ordinary level of skill in the art in view of this description, the claims appended hereto, and the applications incorporated by reference herein.

### BRIEF DESCRIPTION OF THE DRAWINGS

FIG. 1 is a bar graph showing the effects of MTDR on 3D mammosphere formation in MCF7 cells.

FIG. 2 is a bar graph showing the effects of MTDR on 3D mammosphere formation in MDA-MB-231 cells.

FIG. 3 shows the effects of MTDR on 3D mammosphere formation in MDA-MB-231 cells.

FIGS. 4A-4D show the metabolic flux analysis results in MCF7 cells, including OCR, basal respiration, maximal respiration, and ATP production, respectively.

FIGS. 5A-5D show the metabolic flux analysis results in MDA-MB-231 cells, including OCR, basal respiration, maximal respiration, and ATP production, respectively.

FIGS. 6A-6D show the metabolic flux analysis results in MDA-MB-468 cells, including OCR, basal respiration, maximal respiration, and ATP production, respectively.

FIGS. 7A-7D show the results of glycolytic function in MCF7 cells, including ECAR, glycolysis, glycolytic capacity, and glycolytic reserve, respectively.

FIGS. 8A-8D show the results of glycolytic function in MDA-MB-231 cells, including ECAR, glycolysis, glycolytic capacity, and glycolytic reserve, respectively.

FIGS. 9A-9B show the results of glycolytic function in MDA-MB-468 cells, including ECAR, glycolysis, glycolytic capacity, and glycolytic reserve, respectively.

FIG. 10 shows cell viability data for MCF7, MDA-MB-231 and MDA-MB-468 cell monolayers treated with MTDR.

FIGS. 11A-C show mammosphere formation assay results for HTIC, DDI, and IR-780, respectfully.

FIGS. 12A-C show basal respiration, maximal respiration, and ATP production results for metabolic flux analysis of adherent MCF7 cells were treated with HITC.

FIGS. 13A-C show the results of glycolytic function analysis for the HITC treatments, respectively, basal glycolysis, induced glycolysis, and compensatory glycolysis.

FIGS. 14A-C show basal respiration, maximal respiration, and ATP production results for metabolic flux analysis of adherent MCF7 cells were treated with DDI.

FIGS. 15A-C show the results of glycolytic function analysis for DDI treatments on MCF7 cells, respectively, basal glycolysis, induced glycolysis, and compensatory glycolysis.

FIGS. 16A-16G show results from the mammosphere formation assay, for the NHS Ester, Azide, Alkyne, Amine, Maleimide, Alkyne, Hydrazide, and Carboxylic acid Cy5 analogs.

### DESCRIPTION

The following description illustrates embodiments of the present approach in sufficient detail to enable practice of the present approach. Although the present approach is described with reference to these specific embodiments, it should be appreciated that the present approach can be embodied in different forms, and this description should not be construed as limiting any appended claims to the specific embodiments set forth herein. Rather, these embodiments are provided so that this disclosure will be thorough and complete, and will fully convey the scope of the present approach to those skilled in the art.

This description uses various terms that should be understood by those of an ordinary level of skill in the art. The following clarifications are made for the avoidance of doubt. The terms “treat,” “treated,” “treating,” and “treatment” include the diminishment or alleviation of at least one symptom associated or caused by the state, disorder or disease being treated, in particular, cancer. In certain embodiments, the treatment comprises diminishing and/or alleviating at least one symptom associated with or caused by the cancer being treated, by the compound of the invention. In some embodiments, the treatment comprises causing the death of a category of cells, such as CSCs, of a particular cancer in a host, and may be accomplished through preventing cancer cells from further propagation, and/or inhibiting CSC function through, for example, depriving such cells of mechanisms for generating energy. For example, treatment can be diminishment of one or several symptoms of a cancer, or complete eradication of a cancer. As another example, the present approach may be used to inhibit mitochondrial metabolism in the cancer, eradicate (e.g., killing at a rate higher than a rate of propagation) CSCs in the cancer, eradicate TICs in the cancer, eradicate circulating tumor cells in the cancer, inhibit propagation of the cancer, target and inhibit CSCs, target and inhibit TICs, target and inhibit circulating tumor cells, prevent (i.e., reduce the likelihood of) metastasis, prevent recurrence, sensitize the cancer to a chemotherapeutic, sensitize the cancer to radiotherapy, sensitize the cancer to phototherapy.

The terms “cancer stem cell” and “CSC” refer to the subpopulation of cancer cells within tumors that have capabilities of self-renewal, differentiation, and tumorigenicity when transplanted into an animal host. Compared to “bulk” cancer cells, CSCs have increased mitochondrial mass, enhanced mitochondrial biogenesis, and higher activation of mitochondrial protein translation. As used herein, a “circulating tumor cell” is a cancer cell that has shed into the vasculature or lymphatics from a primary tumor and is carried around the body in the blood circulation. The CellSearch Circulating Tumor Cell Test may be used to detect circulating tumor cells.

The phrase “pharmaceutically effective amount,” as used herein, indicates an amount necessary to administer to a host, or to a cell, tissue, or organ of a host, to achieve a therapeutic result, such as regulating, modulating, or inhibiting protein kinase activity, e.g., inhibition of the activity of a protein kinase, or treatment of cancer. A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

Cyanine dyes accumulate in cells derived from solid tumors, e.g., prostate, gastric, kidney, hepatocytes, lung cancer, and glioblastoma, but not in healthy cells in vitro. Cyanine dyes preferentially target mitochondria in cancer cells, by generating a selective chemically-induced cytotoxicity, through redox-based mechanisms. In addition, in vivo experiments have shown that MR cyanine derivatives (e.g., IR-780) in general are safe to use, with a short-term accumulation and a half-life in serum of minutes to hour, whereas, in tumors its fluorescent signal persists for days in animals. In addition, the thiol reactive chloro-methyl moiety (a meso-chlorine-group) increased IR-780 tumor localization in vivo. However, these compounds have been used for theranostic approaches, as well as for photodynamic and photothermal therapy.

The present approach involves the suitability of cyanine compounds, and in particular heptamethine cyanine compounds, as mitochondrial inhibitors having anti-cancer activity. Cyanine compounds have the general formula R₂N[CH=CH]ₙCH=N+R₂↔R₂N⁺=CH[CH=CH]ₙNR₂, wherein ‘R’ may be a variety of groups, and ‘n’ is an integer (normally 2 to 7) in which the nitrogen and part of the conjugated chain may than part of a heterocyclic system, for example, imidazole, pyridine, pyrrole, quinoline and thiazole. Heptamethine cyanine compounds have 7 methine groups extending between nitrogen atoms, and are often referred to using “Cy5” to denote the base heptamethine structure.

In some embodiments of the present approach, the heptamethine cyanine compound is 1-{4-[(chloromethyl)phenyl]methyl}-3,3-dimethyl-2-[5-(1,3,3-trimethyl-1,3-dihydro-2H-indol-2-ylidene)penta-1,3-dien-1-yl]-3H-indolium chloride, otherwise known as MitoTracker Deep Red (MTDR), a well-known mitochondrial fluorescent probe that may be used for targeting mitochondria and effectively inhibiting the propagation of breast cancer stem cells. MTDR is a far-red fluorescent dye that stains active mitochondria and is used as a non-toxic fluorescent chemical probe with a thiol reactive chloromethyl moiety for visualizing the distribution of mitochondria in living cells, and to quantitate mitochondrial potential by FACS or fluorescent microscopy analysis. MTDR is a lipophilic cation, which is a chemical characteristic that increases its efficiency in targeting mitochondria. The chemical structure for MTDR is shown below.

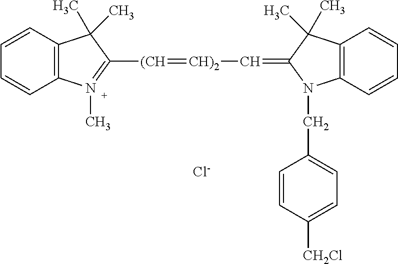

Originally, MTDR was designed for use as a probe to measure mitochondrial mass, independently of mitochondria activity or membrane potential. However, recent experiments directly show that MTDR staining is prevented and/or reduced by treatment with FCCP (carbonyl cyanide 4-(trifluoromethoxy) phenylhydrazone), a potent mitochondrial uncoupling agent. In contrast, MitoTracker Green (MTU) staining remained unchanged during FCCP treatment. Therefore, MTDR may preferentially accumulate in highly active mitochondria, potentially making it a better therapeutic drug for targeting and inhibiting mitochondrial function.

As described herein, MTDR is one of numerous cyanine compounds that target mitochondria in CSCs, and prevent CSC anchorage-independent propagation. This activity is demonstrated using three independent breast cancer cell lines, namely MCF7, MDA-MB-231 and MDA-MB-468 cells, representing both ER(±) and triple-negative breast cancer sub-types. MTDR potently inhibited the 3D propagation CSCs from all three cancer cell lines, even at nano-molar concentrations. Furthermore, analysis using the Seahorse XFe96 metabolic flux analyzer directly validated that MTDR specifically targeted mitochondrial metabolism and induced ATP depletion.

The inventors have previously shown that other lipophilic cations, such as certain derivatives of triphenyl-phosponium (TPP), are effective for targeting mitochondria in CSCs, significantly preventing 3D mammosphere formation. However, compared to many of the cyanine compounds described herein, these TPP derivatives were much less potent, inhibiting 3D spheroid formation in MCF7 cells, with an IC-50 between 500 nM to 5 μM. Therefore, MTDR is approximately 10 to 50-fold more potent than these TPP-derivatives, such as 2,4-dichlorobenzyl-TPP, 1-naphthylmethyl-TPP, 3-methylbenzyl-TPP, 2-chlorobenzyl-TPP, and 2-butene-1,4-bis-TPP. As such, MTDR is more potent and efficacious.

Under the present approach, various analogs of MTDR may be mitochondrial inhibitors and selectively target CSCs. The chemical structure below, formula [A], is a general formula for MTDR analogs. The functional groups R¹ through R¹⁴ represent the positions at which MTDR may be modified and optimized, e.g., to enhance the compound's anti-CSC activity, via medicinal chemistry.

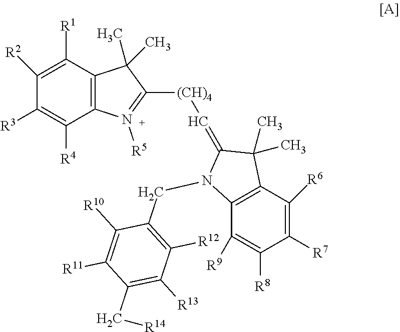

In formula [A], each of R¹ through R¹⁴ may be the same or different, and may selected from hydrogen, carbon, nitrogen, sulfur, oxygen, fluorine, chlorine, bromine, iodine, carboxyl, alkanes, cyclic alkanes, alkane-based derivatives, alkenes, cyclic alkenes, alkene-based derivatives, alkynes, alkyne-based derivative, ketones, ketone-based derivatives, aldehydes, aldehyde-based derivatives, carboxylic acids, carboxylic acid-based derivatives, ethers, ether-based derivatives, esters and ester-based derivatives, amines, amino-based derivatives, amides, amide-based derivatives, monocyclic or polycyclic arene, heteroarenes, arene-based derivatives, heteroarene-based derivatives, phenols, phenol-based derivatives, benzoic acid, benzoic acid-based derivatives, membrane-targeting signals, and mitochondria-targeting signals, provided that at least one of R¹ through R¹⁴ is not H.

In some embodiments, one or more R groups may comprise a targeting signal to further increase the mitochondrial uptake of the carbocyanine compound. For examples of targeting signals, including membrane-targeting signals and mitochondrial-targeting signals, see, for example, the approaches disclosed in International Patent Application PCT/US2018/033466, filed May 18, 2018, International Patent Application PCT/US 2018/062174, filed Nov. 21, 2018, and International Patent Application PCT/US2018/062956, filed Nov. 29, 2019, each of which is incorporated herein by reference in its entirety. The addition of one or more targeting signals to a carbocyanine compound can significantly increase the effectiveness of that compound, in some instances by over 100 times in the target organelle. Such modification may allow for smaller concentrations or doses, another advantageous benefit of the present approach.

One or more R-groups may comprise a membrane-targeting signal. Examples of membrane-targeting signals include palmitic acid, stearic acid, myristic acid, oleic acid, short chain fatty acids (i.e., having 5 or fewer carbon atoms in the chemical structure), medium-chain fatty acids (having 6-12 carbon atoms in the chemical structure). As an example, one of R¹ through R¹⁴ may be a fatty acid moiety, such as a myristate. One or more R-groups may comprise a membrane-targeting signal. Examples of mitochondria-targeting signals include lipophilic cations such as tri-phenyl-phosphonium (TPP), TPP-derivatives, guanidinium, guanidinium derivatives, and 10-N-nonyl acridine orange. It should be appreciated that these examples are not intended to be exhaustive. MTDR, like many carbocyanine compounds, is already a lipophilic cation, and as such it preferentially targets cellular mitochondria. Even so, some embodiments experience improved targeting with the addition of a lipophilic cation.

In addition to MTDR and its analogs, other NIR dyes are shown to inhibit CSC growth in MCF7 cells. These include HITC iodide, DDI, and IR-780. The structure for these compounds are shown below. The data show that MTDR, HITC and DDI are all effective inhibitors of MCF7 CSC growth. However, IR-780 had no significant effect in the nanomolar range. In addition to these demonstrative compounds, seven Cyanine 5 (Cy5) heptamethine analogs with different reactive groups were examined for their ability to inhibit CSC growth. Overall, compounds identified as Cy5-Azide and Cy5-Alkyne, described below, are both effective inhibitors of CSCs, in the nanomolar range. It should be appreciated that other carbocyanine compounds may have similar efficacy, and efforts are underway to identify other carbocyanine compounds, including derivatives of MTDR, that may be used in the present approach. Further analysis of other cyanine compounds, including several described herein at higher concentrations, are underway.

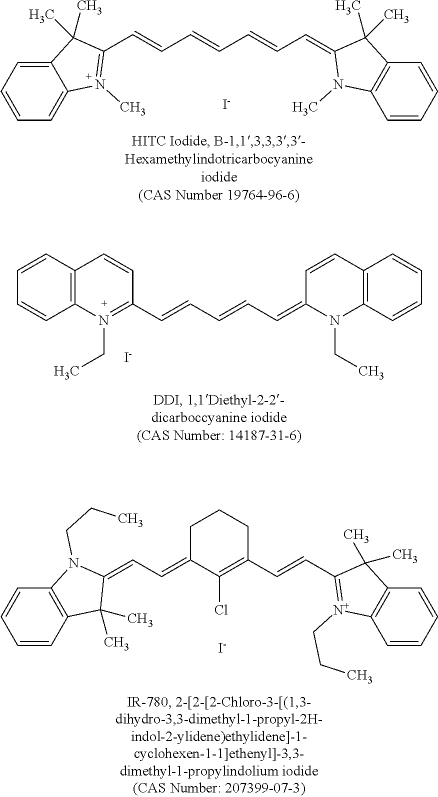

The suitability of cyanine compounds for targeting mitochondria and effectively inhibiting the propagation of breast CSCs were investigated using a variety of assays and three primary model cell lines: MCF7, MDA-MB-231 and MDA-MB-468. MCF7 is an ER(+) breast cancer cell line, while MDA-MB-231 and MDA-MB-468 are both considered triple negative [ER(−), PR(−), HER2(−)] cell lines. In this context, the inventors assessed the targeted effects of MTDR on 3D CSC propagation and overall metabolic rates in monolayer cultures.

MTDR inhibits the 3D anchorage-independent propagation of CSCs. In order to assess the effects of MTDR on CSC propagation, the mammosphere assay was used as a functional readout of “stemness” and 3D anchorage-independent growth. As CSCs are highly-resistant to many types of cell stress, they can undergo anchorage-independent propagation, under low-attachment conditions. Ultimately, this results in the generation of >50 μM sized 3D spheroid-like structures. These “mammospheres” are highly enriched in CSCs and progenitor-like cells, and highly resemble the morula stage of embryonic development, a solid ball of cells without a hollow lumen. Under these culture conditions of non-attachment, the majority of epithelioid cancer cells die, via an unusual form of apoptosis, known as anoikis.

Each single 3D mammosphere is constructed from the anchorage-independent clonal propagation of an individual CSC, and does not involve the process of self-aggregation, under these limiting dilution conditions. As a consequence, the growth of 3D spheroids provides functional culture conditions to select for a population of epithelioid CSCs, with EMT properties. As such this provides an ideal assay for identifying small molecules that can target the anchorage-independent growth of CSCs.

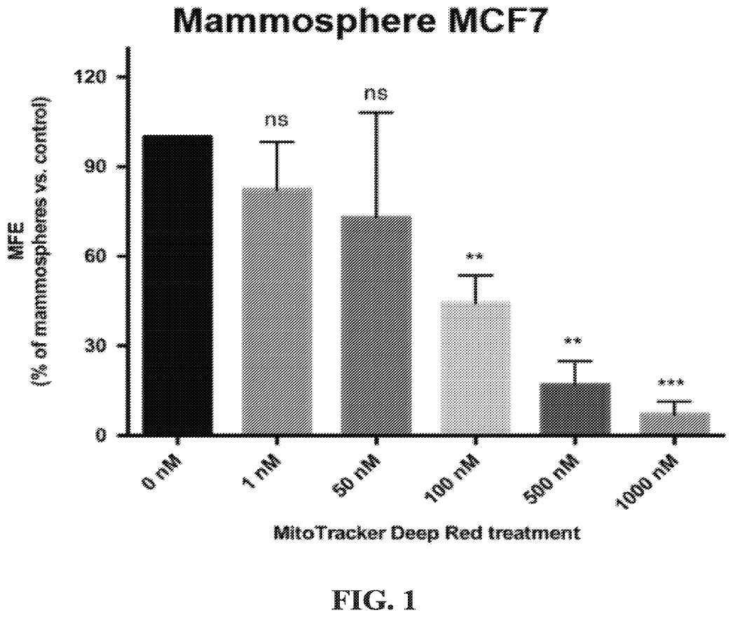

FIG. 1 is a bar graph showing the effects of MTDR on 3D mammosphere formation in MCF7 cells. The mammosphere formation efficiency (MFE) is a relative showing of mammosphere growth relative to a vehicle-only control. The mammosphere formation assay was performed at concentrations of MTDR ranging from 1 nM to 1,000 nM. As can be seen, MTDR inhibits 3D anchorage-independent growth in MCF7 cells with an IC-50 of less than 100 nM.

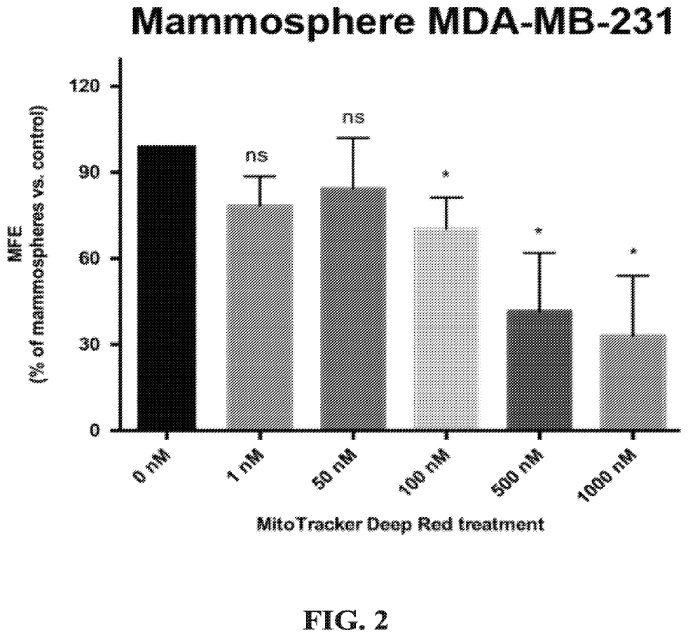

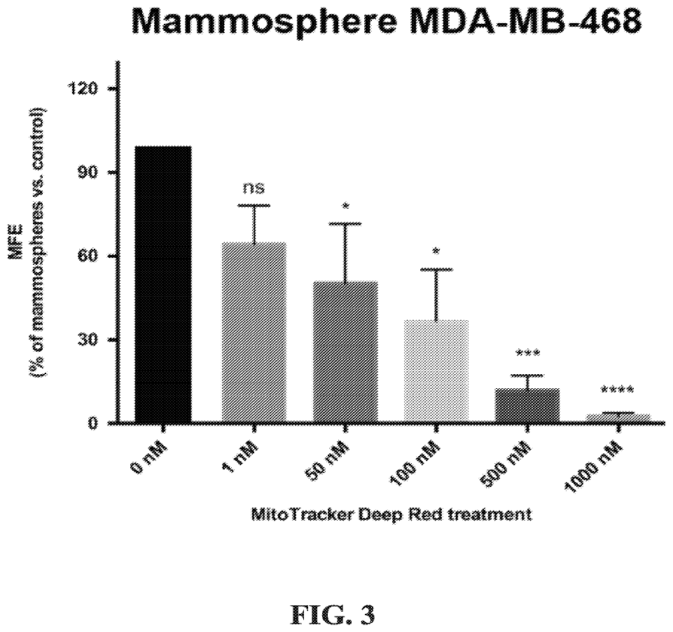

MTDR also inhibited the anchorage-independent growth of MDA-MB-231 cells, at least at concentrations above 100 nM. FIG. 2 is a bar graph showing the effects of MTDR on 3D mammosphere formation in MDA-MB-231 cells. Similar effects can be seen in FIG. 3, which shows the results of the mammosphere formation assay on MDA-MB-468 cells. MTDR inhibited 3D sphere formation in MDA-MB-468 cells with an IC-50 of approximately 50 nM. These results demonstrate that MTDR is effective in targeting CSCs, in both ER(+) and triple-negative breast cancer-derived cell lines. Advantageously, these effects are present at concentrations in the nano-molar range.

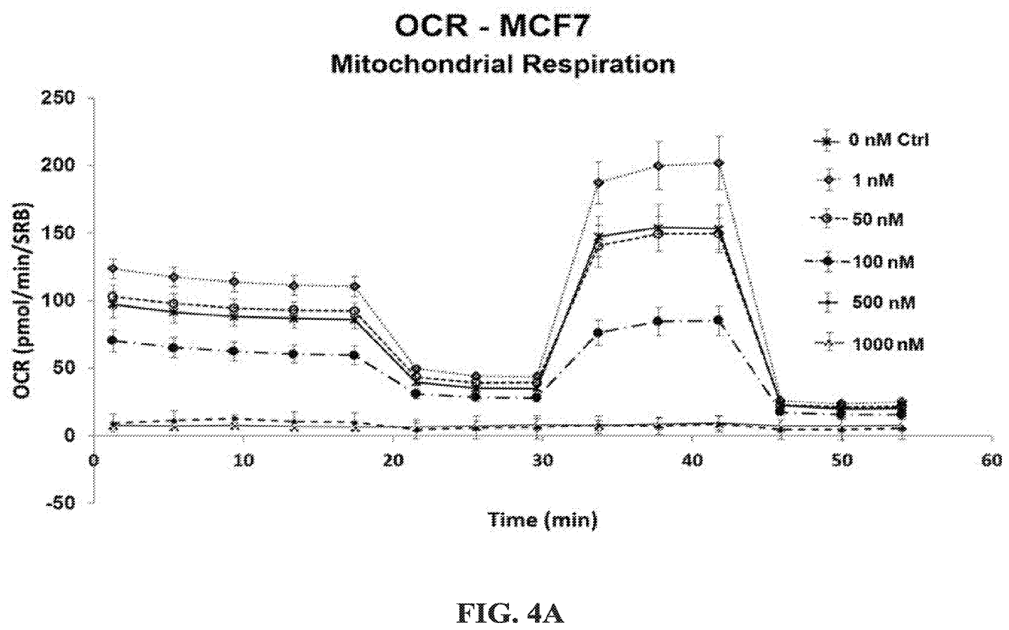

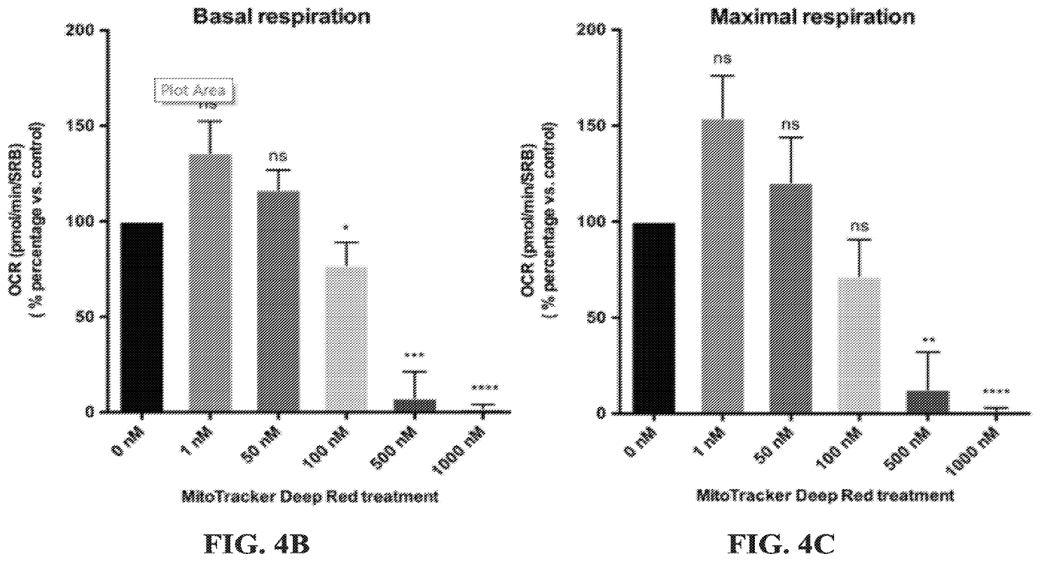

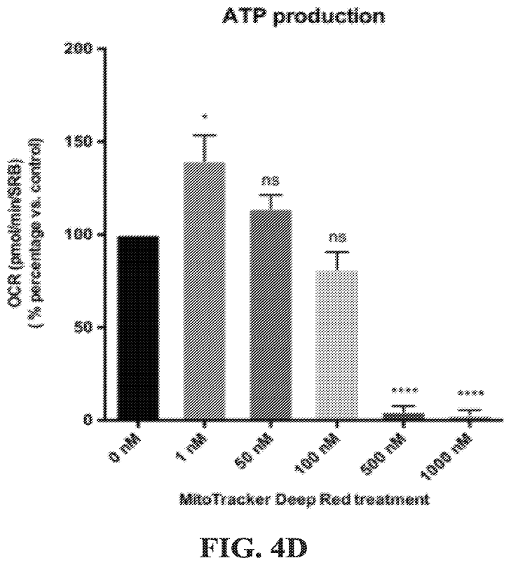

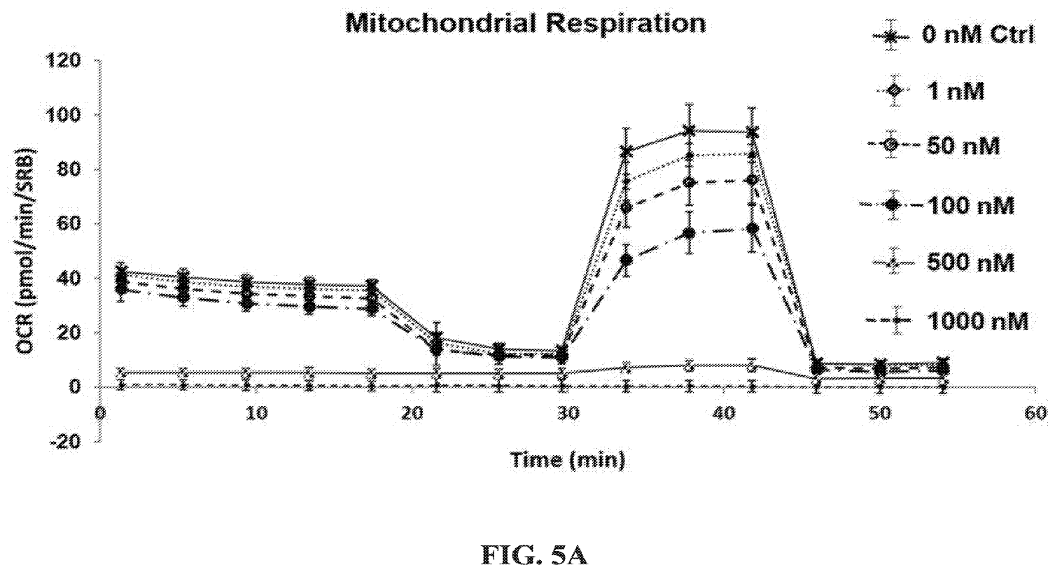

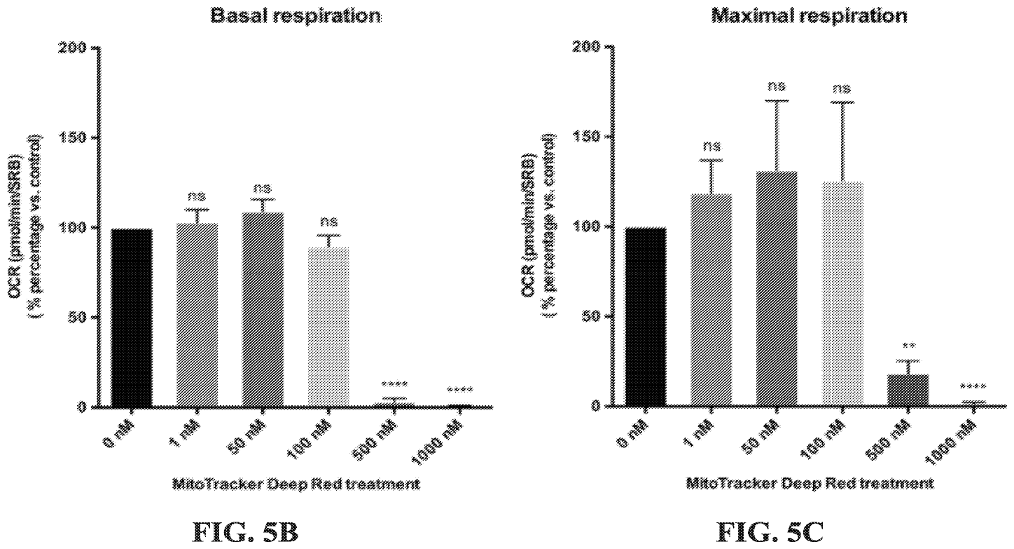

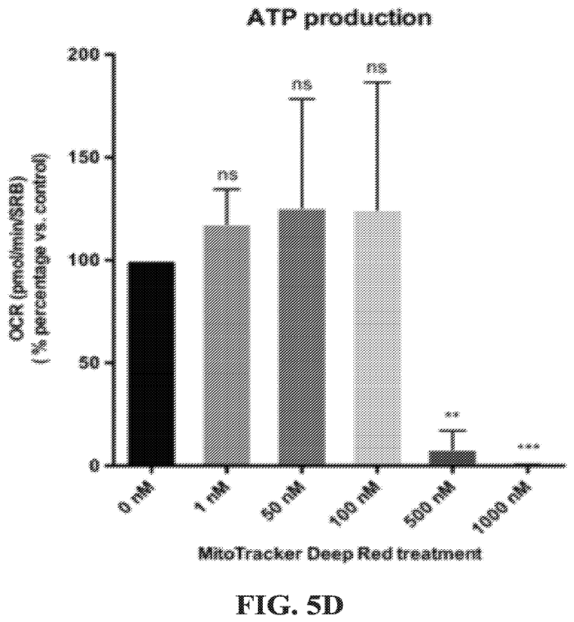

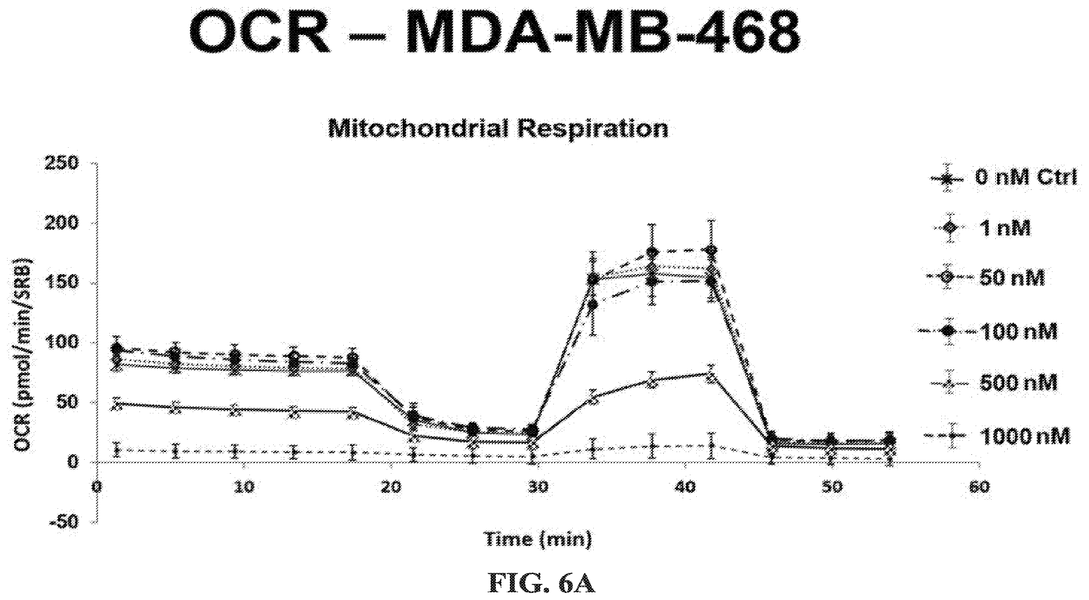

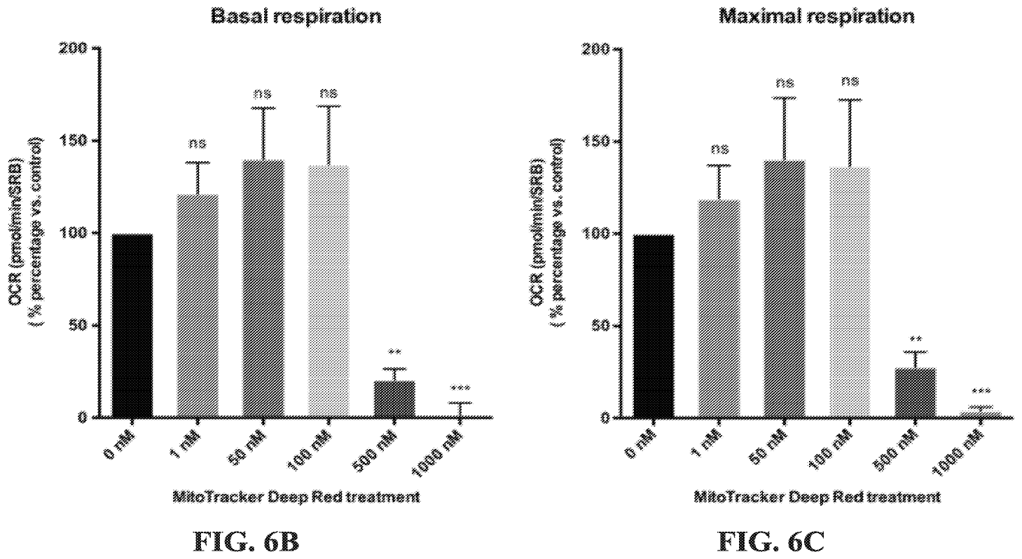

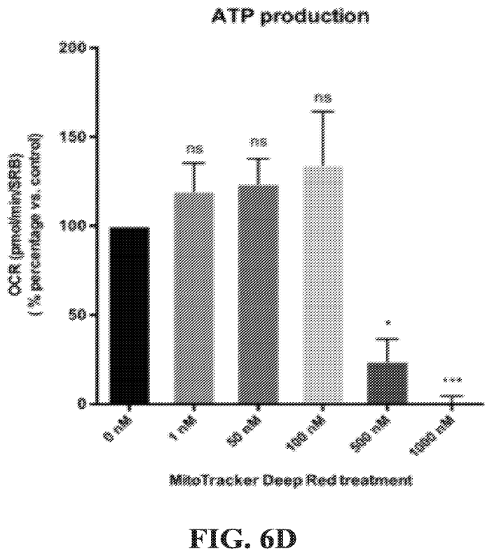

MDR's anti-cancer effect is due (at least in part) to the compound's mitochondrial metabolism inhibition activity. This activity was demonstrated through metabolic flux analysis on monolayer cultures, using the Seahorse XFe96. FIGS. 4A-4D show the metabolic flux analysis results in MCF7 cells, FIGS. 5A-5D show the metabolic flux analysis results in MDA-MB-231 cells, and FIGS. 6A-6D show the metabolic flux analysis results in MDA-MB-468 cells. FIGS. 4A, SA, and 6A show representative Seahorse tracings, while the FIGS. 4B-4D, 5B-5D, and 6B-6D are bar graphs highlighting the quantitative, dose-dependent effects of MTDR on basal respiration, maximal respiration and ATP production.

The impact of MTDR on mitochondrial OCR is evident in all three cell lines. As can be seen, MTDR treatment induced near complete inhibition of mitochondrial function and ATP production, starting at a concentration of 500 nM. MTDR potently inhibits the mitochondrial OCR in MCF7 cells.

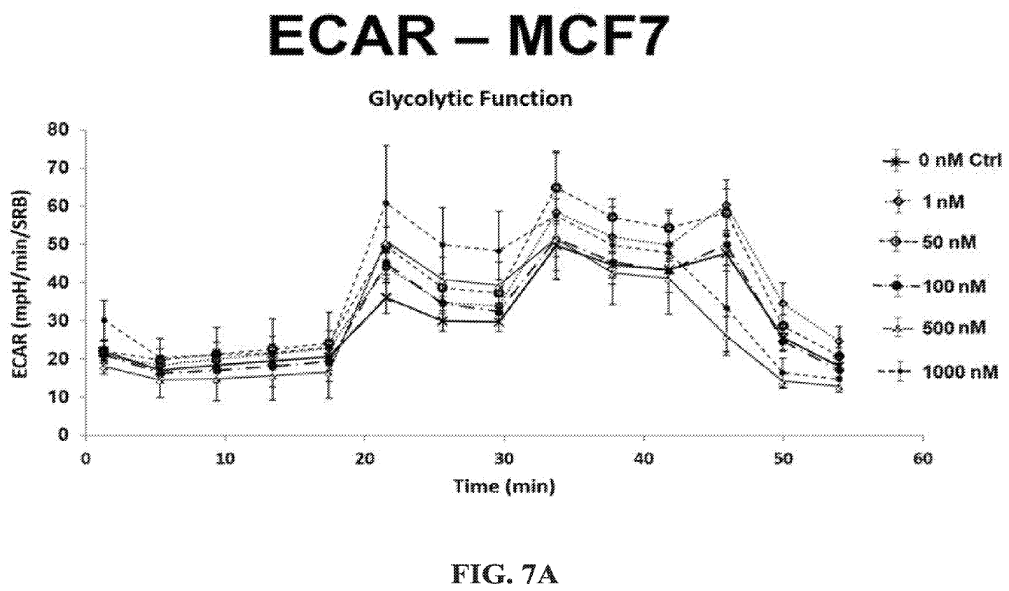

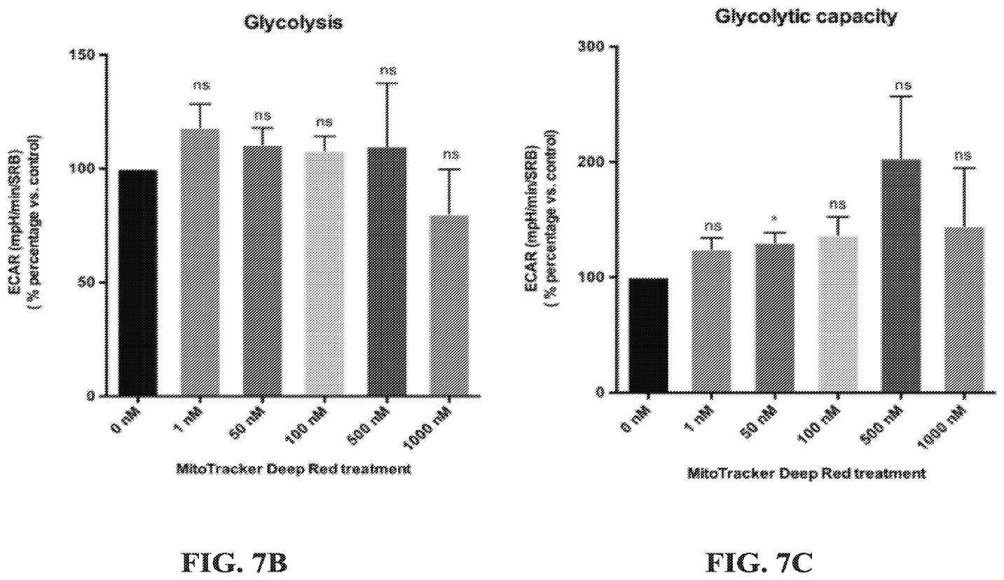

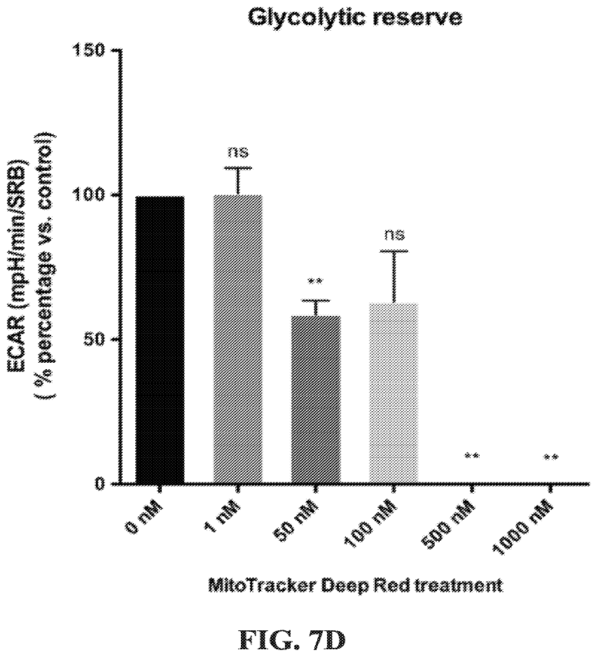

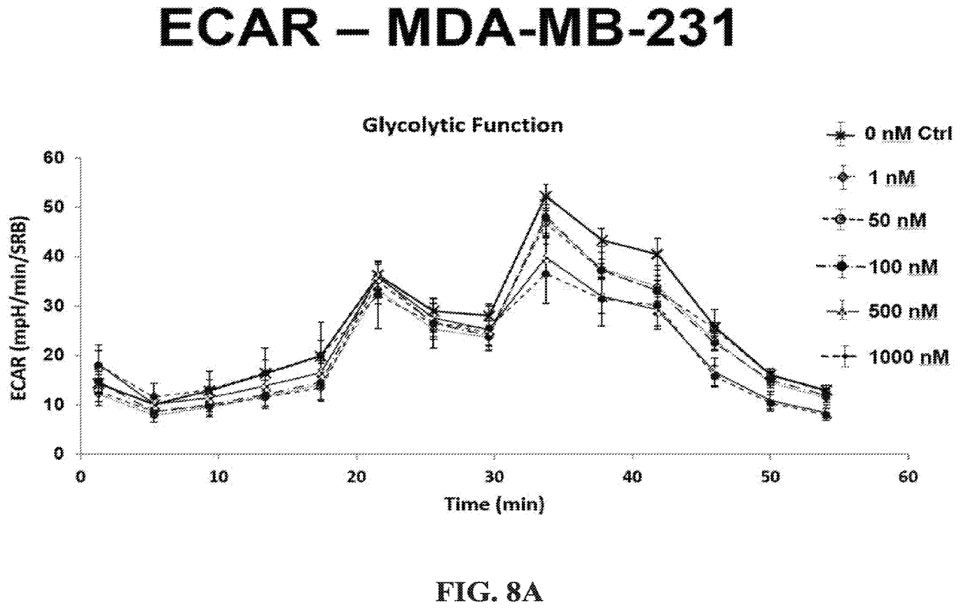

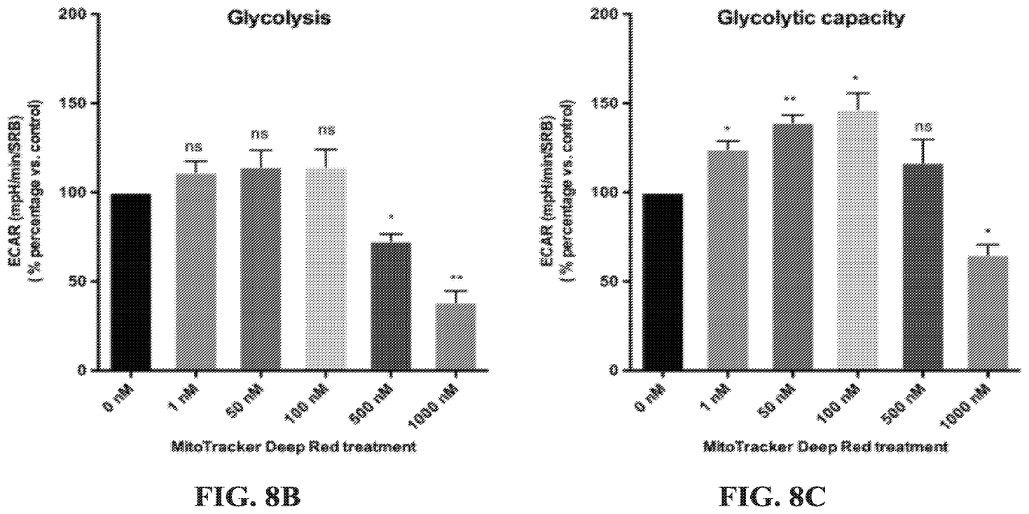

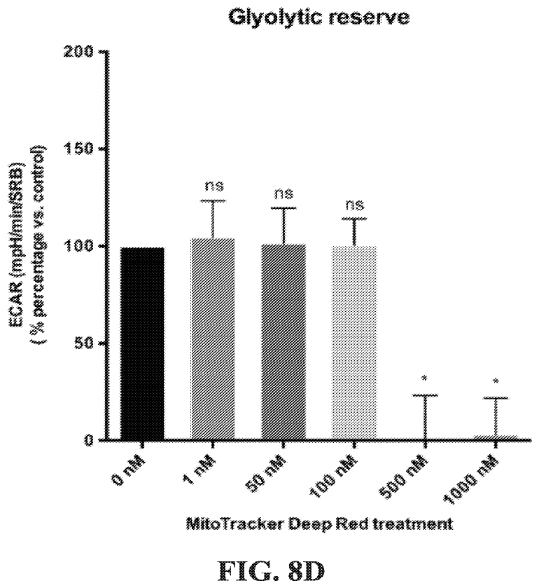

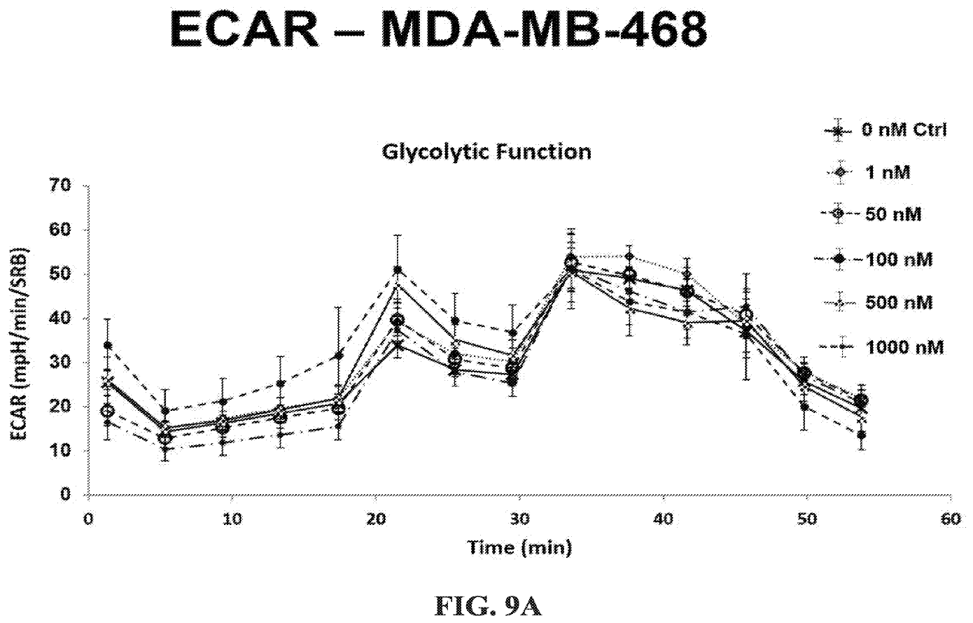

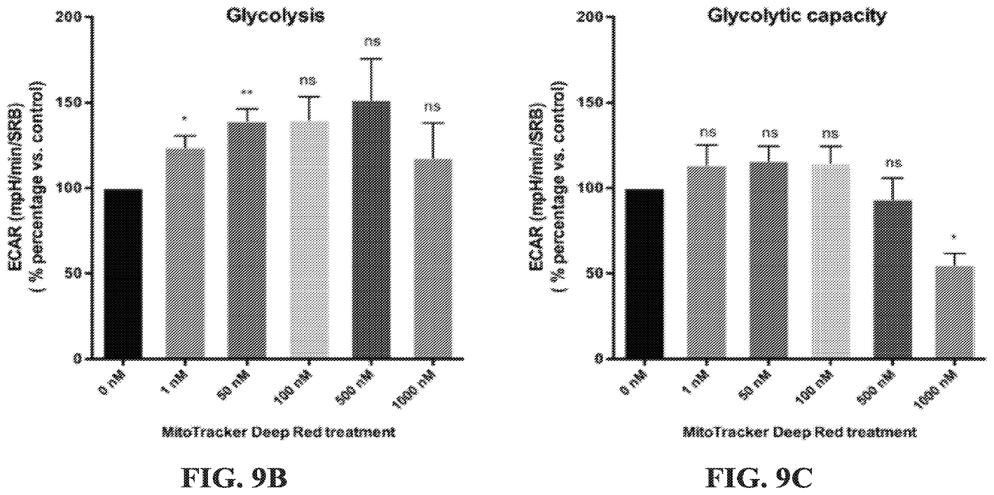

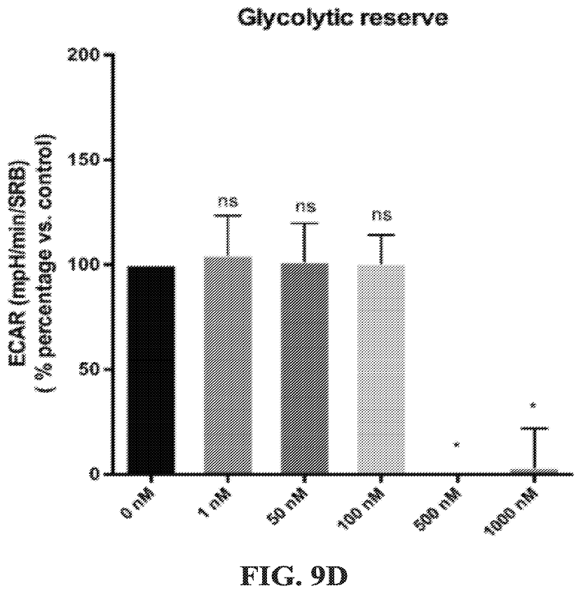

In addition to metabolic flux analysis, glycolytic function was analyzed at different concentrations of MTDR. This included extracellular acidification rate (ECAR) measurements, glycolysis, glycolytic capacity, and glycolytic reserve. FIGS. 7A-7D show the results of glycolytic function in MCF7 cells, including ECAR, glycolysis, glycolytic capacity, and glycolytic reserve, respectively. FIGS. 8A-8D show the results of glycolytic function in MDA-MB-231 cells, including ECAR, glycolysis, glycolytic capacity, and glycolytic reserve, respectively. FIGS. 9A-9D show the results of glycolytic function in MDA-MB-468 cells, including ECAR, glycolysis, glycolytic capacity, and glycolytic reserve, respectively.

The data show that MTDR has no effect on glycolysis in MCF7 cells or MDA-MB-468 cells, but minor effects on glycolysis in MDA-MB-231 cells. A representative Seahorse tracing is shown in FIGS. 7A, 8A, and 9A. These figures show that the ECAR, a measure of glycolytic function, remained largely unchanged in MCF7 and MDA-MB-468 cell monolayers, at levels of MTDR of up to 1 μM. The bar graphs shown in FIGS. 7B-7D, 8B-8D, and 9B-9D, show the quantitative, dose-dependent effects of MTDR on glycolysis, glycolytic capacity and glycolytic reserve for each cell type. It can be seen that MTDR has no significant effect on glycolysis, at concentrations up to 1 μM for MCF7 cells and MDA-MB-231 cells, and for MDA-MB-231 cells MTDR showed no significant effect on glycolysis at concentrations up to 100 nM, and mild-to-moderate inhibition of glycolysis was only observed, starting at 500 nM. Therefore, high nano-molar concentrations of MTDR, of 500 nM or greater, preferentially affected mitochondrial metabolism in all three breast cancer cell lines tested.

In addition to inhibiting mitochondrial metabolism, MTDR preferentially and selectively targets cancer cells. A Hoechst-based viability assay was used to characterize the selectivity of MTDR for the preferential targeting of cancer cells. Briefly, MCF7, MDA-MB-231 and MDA-MB-468 cell monolayers were treated with MTDR, at concentrations ranging from 1 nM to 1 μM, for a period of one day. Cell viability was assessed using Hoechst 33342, a nuclear dye that stains DNA in live cells. The viability of normal human fibroblasts (hTERT-BJ1) treated with MTDR was also assessed in parallel. Quantitation was performed with a plate-reader.

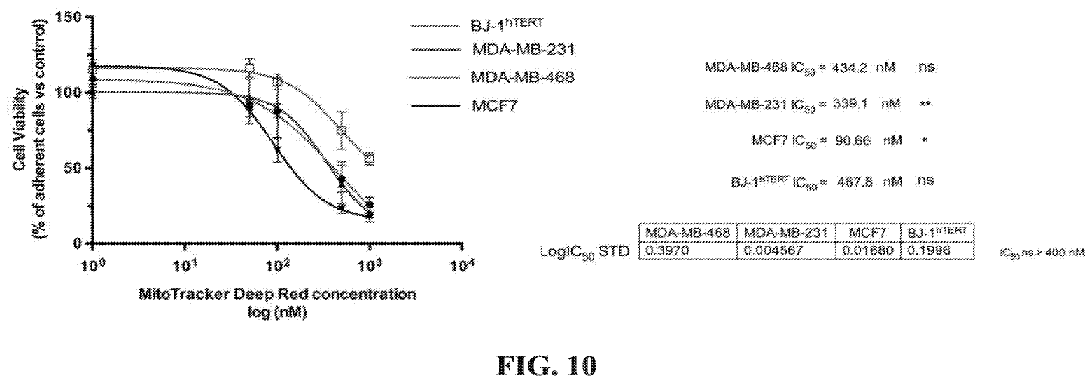

FIG. 10 shows cell viability for MCF7, MDA-MB-231 and MDA-MB-468 cell monolayers treated with MTDR. It can be seen that MTDR effectively killed MCF7 (IC-50=90.66), but was less effective on MDA-MB-231 (IC-50=399.1), MDA-MB-468 (IC-50=432.2) and hTERT-BJ1 (IC-50=467.8). MTDR preferentially and selectively targets cancer cells. MCF7, MDA-MB-231 and MDA-MB-468 cell monolayers were treated with MTDR for a period of 72 hours, and viability was assessed using Hoechst 33342, a nuclear dye that stains DNA in live cells. Effects of MTDR on the viability in normal human fibroblasts (hTERT-BJ1) were assessed in parallel. The results show that MTDR effectively killed MCF7, MDA-MB-231 and MDA-MB-468 cells. The IC-50 for the effects of MTDR on normal cell viability (hTERT-BJ1) was 1 μM. Therefore, MTDR is 10 times more potent and selective for the targeting of breast cancer cells, relative to normal fibroblasts. Further, MTDR was more potent and selective for the targeting ER (+) breast cancer cells and was less effective on triple-negative breast cancer cells, with virtually no effect on normal fibroblast viability.

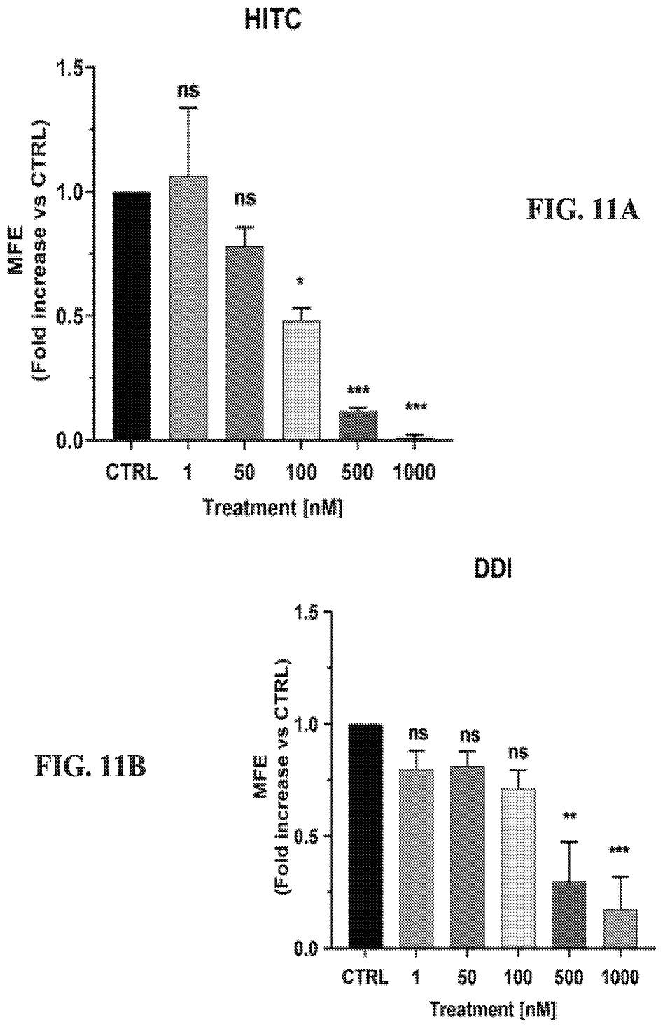

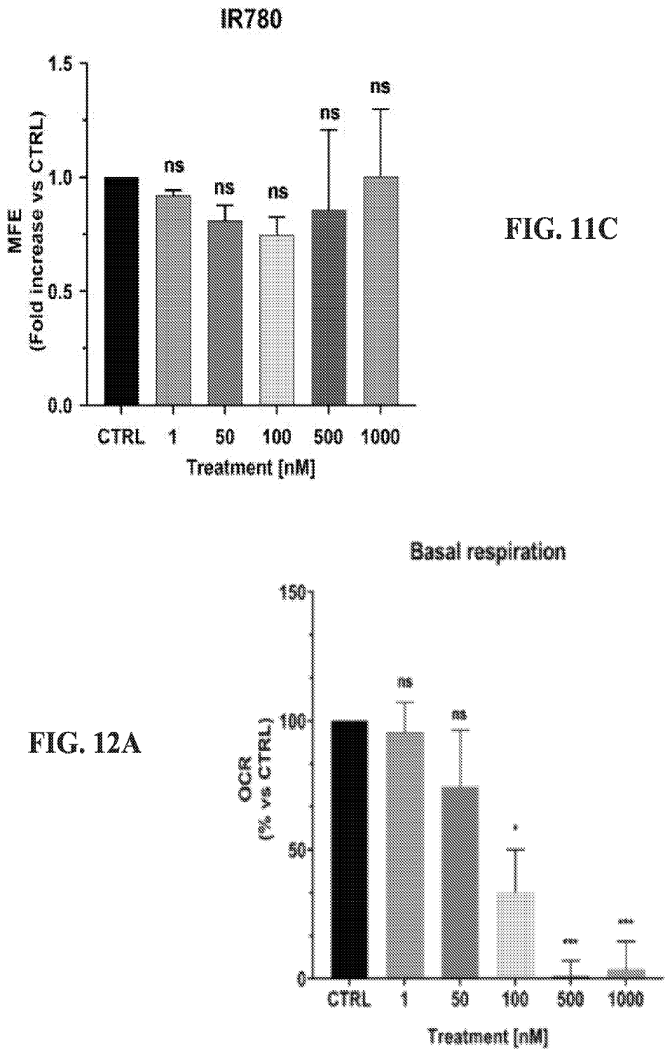

Other near-infrared cyanine compounds with similar spectral emission as MTDR were shown to have anti-cancer activity. The MCF7 3D-mammosphere assay was used to assess the effect of cyanine compounds HITC, DDI, and IR-780, on CSC propagation. The structures for these compounds are shown above. HITC, DDI and IR-780 were tested using the same nanomolar concentration range used for MTDR. The mammosphere assay results for HITC, DDI, and IR-708 are shown in FIGS. 11A-11C, respectfully.

FIGS. 11A-11C show mammosphere formation assay results for HTIC, DDI, and IR-780, respectfully. Briefly, non-adherent MCF7 cells were treated using different drug concentrations of HITC, DDI and IR780 (1, 50, 100, 500, 1000 nM) for five days, and then manually counted. Data is expressed as fold increase versus control. Statistical analysis was conducted using one-way ANOVA (p=0.05). The data shows_illustrates that both HITC and DDI significantly inhibited CSC propagation, between 100 and 1,000 nM. In contrast, IR-780, was not effective. In support of these findings, images of the cells showed that only HITC and DDI were efficiently incorporated into 3D-mammospheres. IR-780, on the other hand, was not taken up by MCF7 CSCs, at concentrations in the nano-molar range.

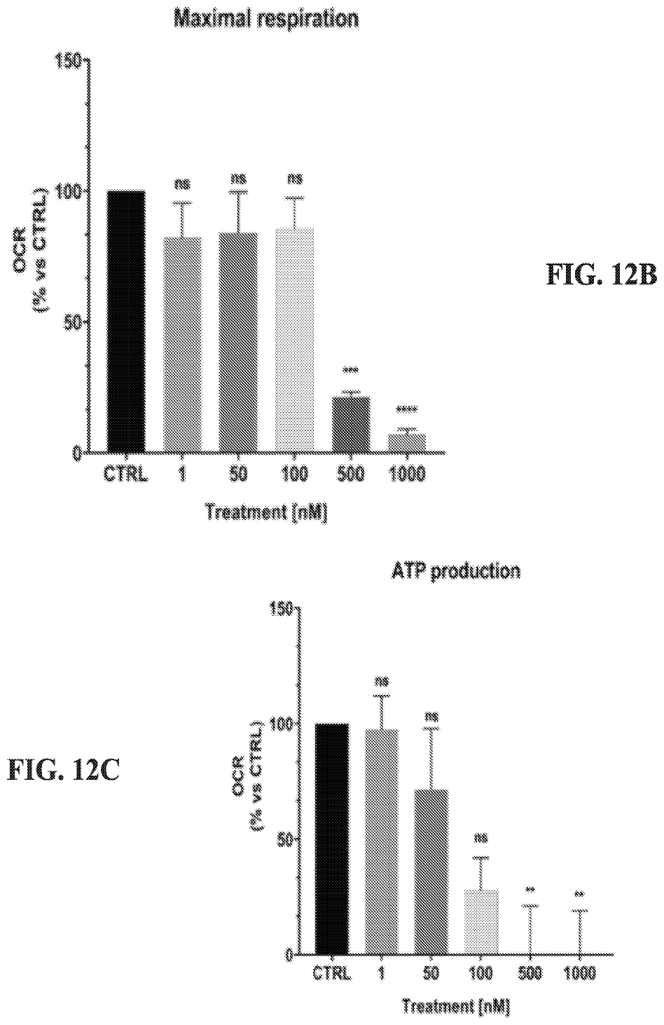

To examine the effects of HITC and DDI on mitochondrial respiration and aerobic glycolysis, adherent MCF7 were treated with each compound, and then OCR and ECAR were measured. FIGS. 12A-C show basal respiration, maximal respiration, and ATP production results for metabolic flux analysis of adherent MCF7 cells were treated with HITC using five different concentrations for 16 hours. After treatment, the mitochondrial oxygen consumption rate (OCR) was measured using the Seahorse XFe96 analyzer. Data is expressed as percentage of OCR versus control. All data was normalized for cell number. Statistical analysis was conducted using one-way ANOVA.

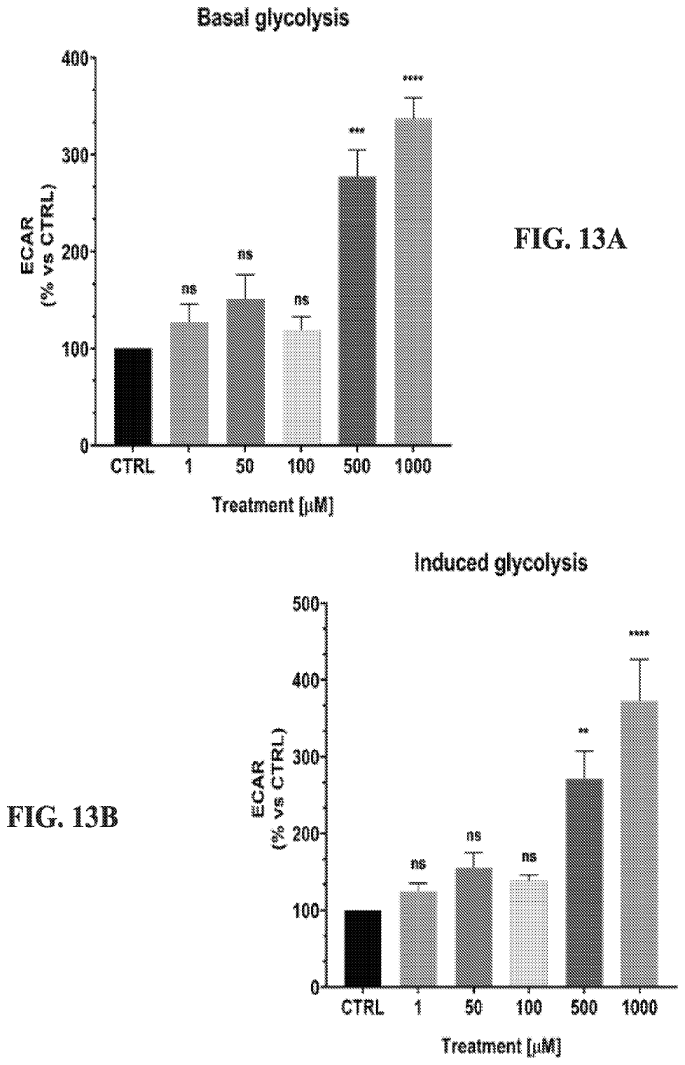

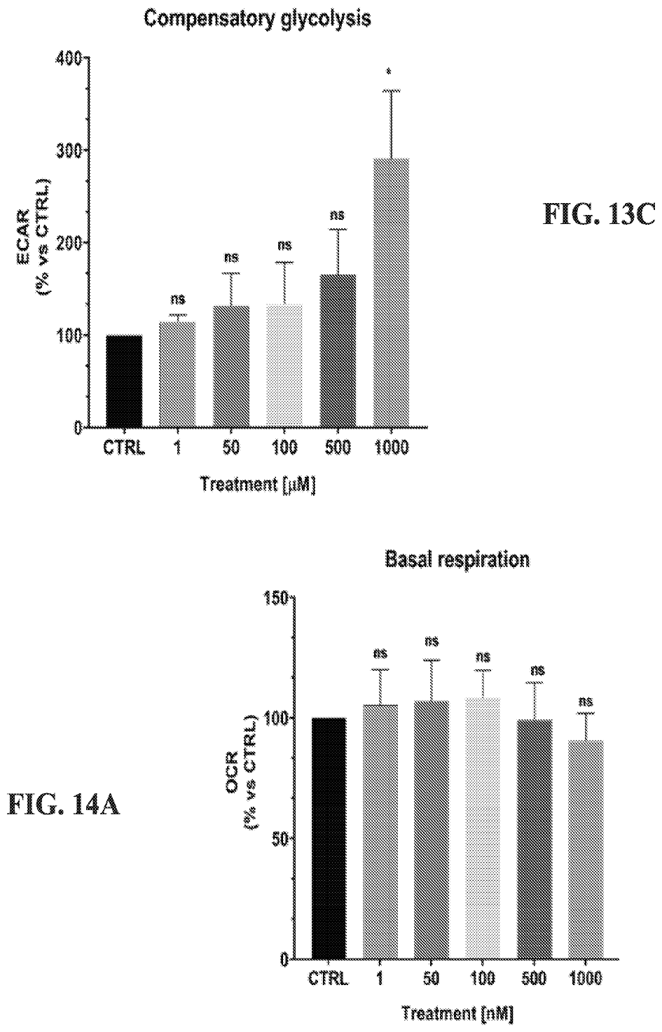

FIGS. 13A-C show the results of glycolytic function analysis for the HITC treatments. Basal glycolysis, induced glycolysis, and compensatory glycolysis, respectively. The data show that HITC significantly inhibited basal and maximal OCR, as well as ATP production levels, as compared to vehicle-alone control cells. In contrast, ECAR levels were increased significantly, at 500 and 1000 nM.

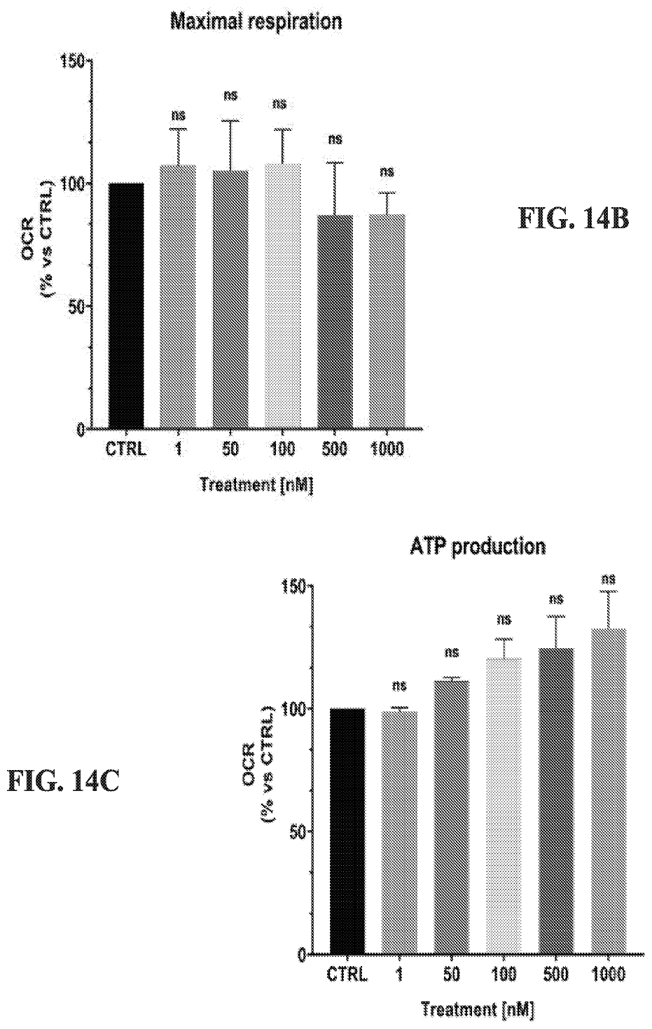

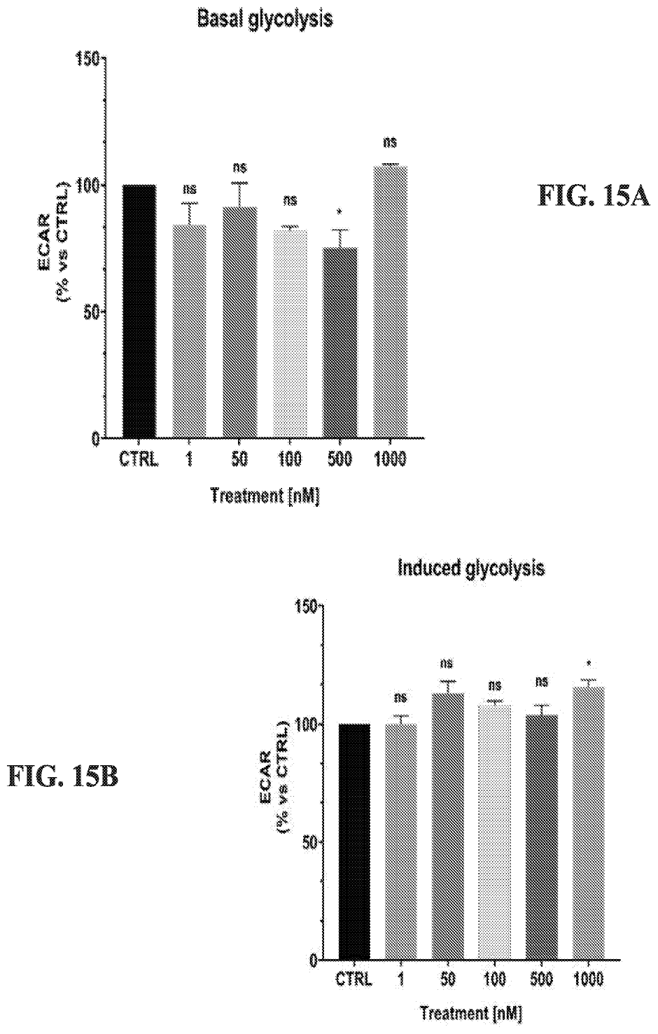

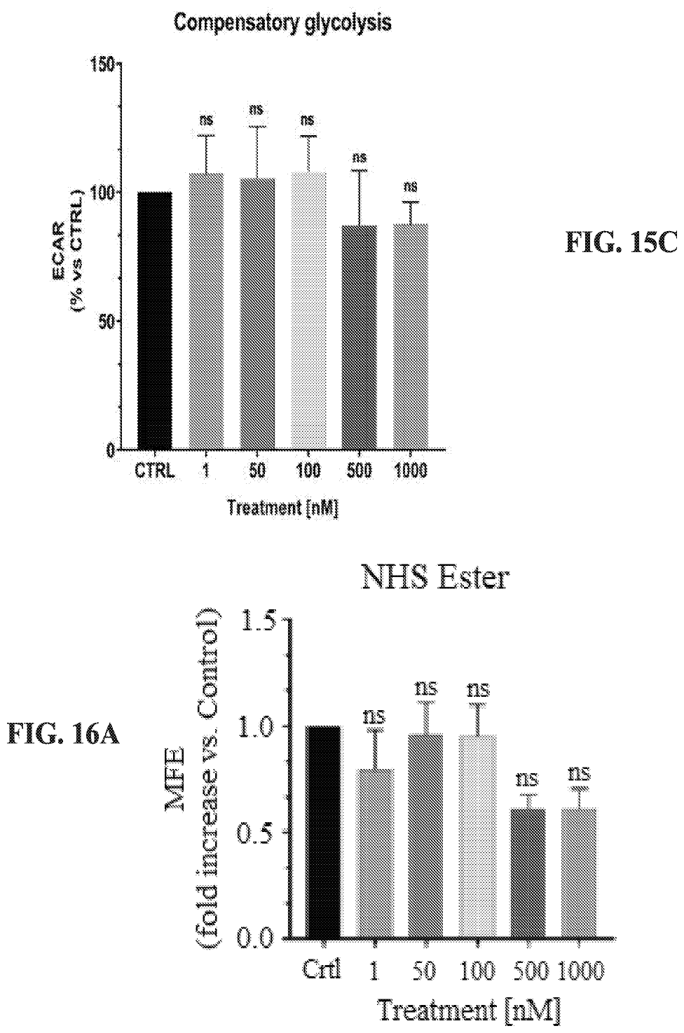

DDI, on the other hand, did not affect OCR or ECAR in MCF7 cells. FIGS. 14A-C show basal respiration, maximal respiration, and ATP production results for metabolic flux analysis of adherent MCF7 cells were treated with DDI. FIGS. 15A-C show the results of glycolytic function analysis for DIN treatments on MCF7 cells, respectively, basal glycolysis, induced glycolysis, and compensatory glycolysis.

These results show that HITC specifically targets mitochondrial metabolism and inhibits 3D-mammosphere formation. In contrast, DDI also inhibits 3D-mammosphere formation, but by a mitochondrial-independent mechanism. Thus, some Finally, IR-780 did not inhibit CSC propagation in the nanomolar range.

The mitochondrial inhibition effect of other Cyanine 5 (Cy5) analogs have been explored. To assess the possible anti-cancer properties of Cy5 lipophilic fluorophores, the potential inhibitory activity of seven commercially available Cy5 analogs were tested, and testing further compounds are underway. The base formula for these compounds is shown below as formula [B]:

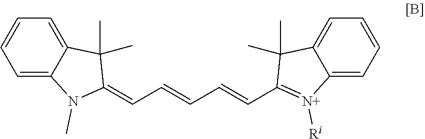

where Rᵢ depends on the particular Cy5 analog. The chemical structure of Cyanine 5 compounds is characterized by a polymethine bridge in between the two nitrogen atoms. The positive charge (+) is delocalized within the scaffold on one of the two amine groups (N+). The amine group can be used to covalently bond several potential side chains. The table below identities Rᵢ for the 7 Cy5 analogs described herein. It should be appreciated that further Cy5 analogs are being evaluated.

| Cy5 Analog | Rᵢ |
|---|---|
| NHS Ester | 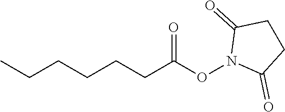 |
| Amine | 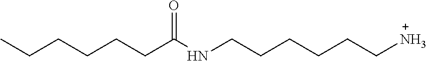 |
| Azide | 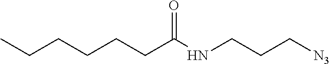 |
| Maleimide | 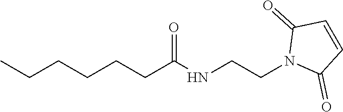 |
| Alkyne | 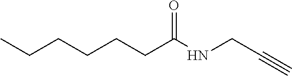 |
| Hydrazide | 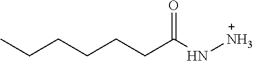 |
| Carboxylic Acid | 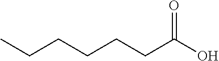 |

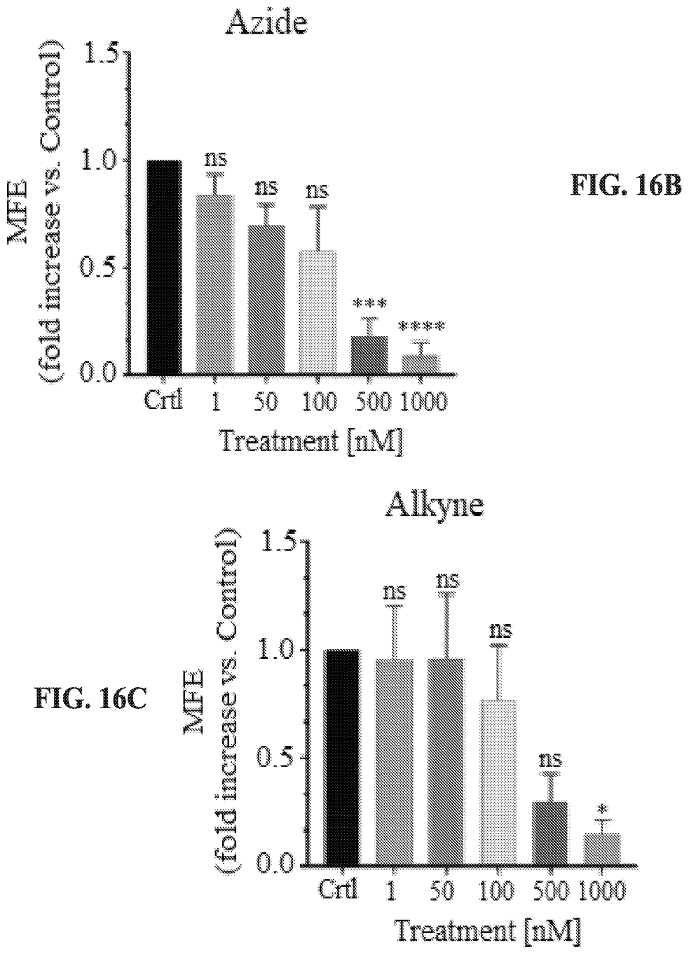

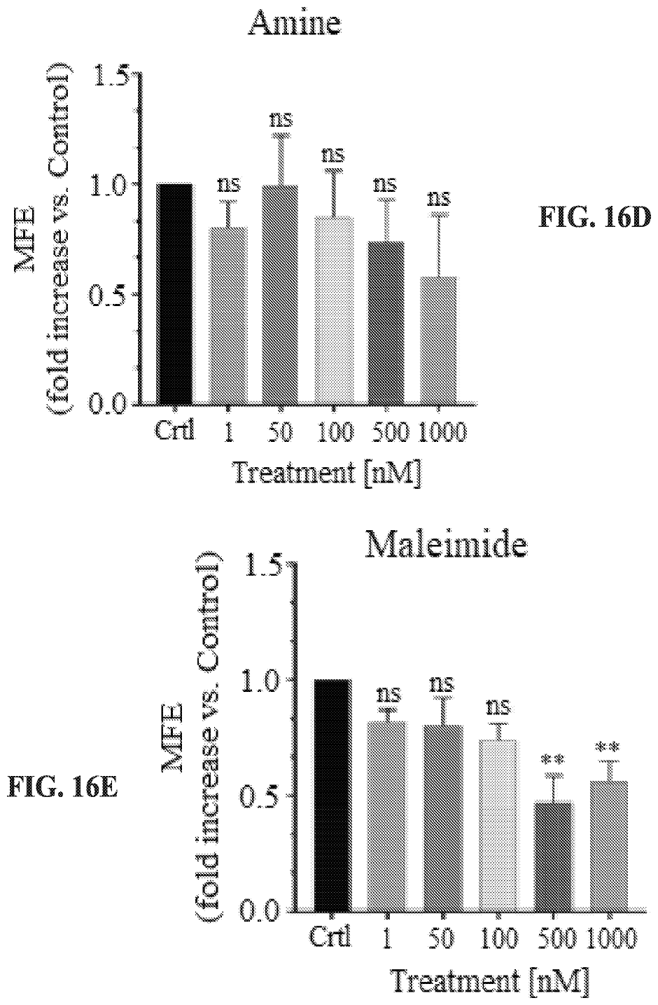

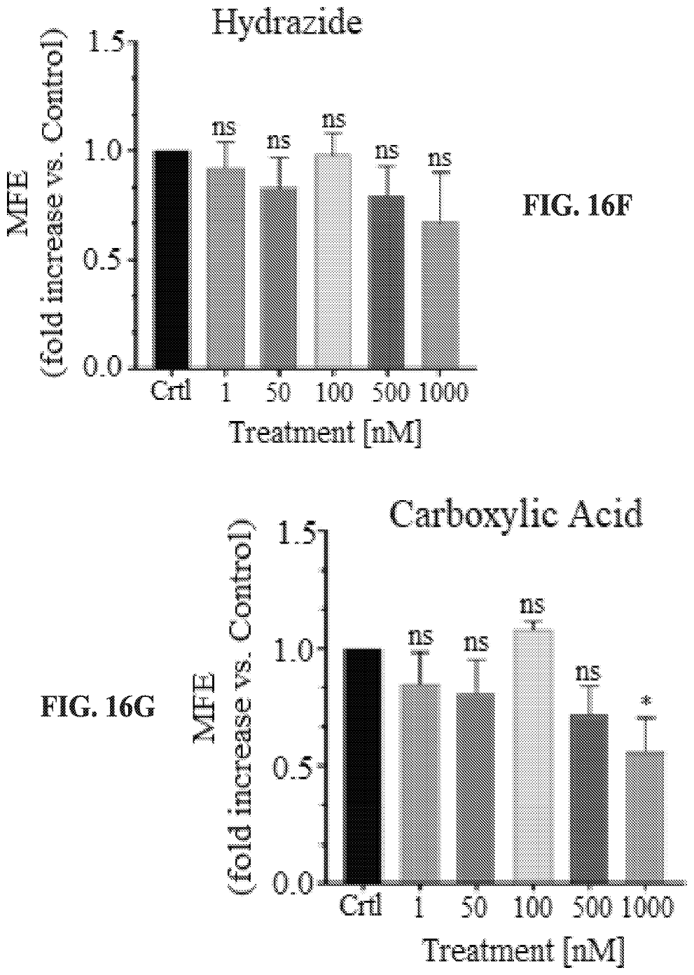

FIGS. 16A-16G show results from the mammosphere formation assay for the NHS Ester, Azide, Alkyne, Amine, Maleimide, Alkyne, Hydrazide, and Carboxylic Acid analogs. Briefly, MCF7 mammospheres cells were treated with different concentrations of each compound (1, 50, 100, 500 and 1000 nM) for five days. Mammospheres above 50<μm were counted manually using a bright field microscope (n=4). Data is expressed as fold increase versus control. Statistical analysis was conducted using one-way ANOVA (p=0.05). The data show that the Azide (Cy5-Azide) and Alkyne (Cy5-Alkyne) analogs were the only two compounds out of the seven analogs tested to significantly inhibit MCF7 3D-mammosphere formation at concentrations between 500 nM to 1000 nM.

However, it was evident from fluorescent image analysis that all of the Cy5 analogs were internalized by the mammospheres, at low nanomolar concentrations (50 nM), independently from their anti-CSC effects. Microscopy analysis of MCF7 dye internalization at a concentration 50 nM for each analog was used, and images were acquired with an EVOS fluorescent microscope, using Cy5 channel and a 20× objective. These results show that Cy5 retention lasts for days in CSCs. Furthermore, both carbocyanine compounds (Cy5-Azide and Cy5-Alkyne) are mitochondrial OXPHOS inhibitors at concentrations ranging from 500 nM and above, and they induce glycolysis to compensate for mitochondrial ATP depletion.

It should be appreciated from the foregoing that cyanine compounds, including MTDR, analogs of MTDR, and certain other Cy5 analogs, can be used effectively as a metabolic inhibitor to target mitochondrial function and halt CSC propagation. MTDR, in particular, is effective as an anti-CSC therapeutic in the nano-molar range. The metabolic effects of MTDR on mitochondrial oxygen consumption rates (OCR) and ATP production have been directly validated, thereby establishing that MTDR is an effective and potent inhibitor of mitochondrial metabolism. Given these properties, in some embodiments of the present approach, MTDR is repurposed as a potent and selective anti-cancer agent, to target the CSC population, in a variety of cancer types. Because of the anti-mitochondrial effects of MTDR, some embodiments may also possess anti-aging activity, radiosensitizing activity, photosensitizing activity, and/or anti-microbial activity. Some embodiments may sensitize cancer cells to chemotherapeutic agents, natural substances, and caloric restriction.

In some embodiments, the present approach targets this dependency through a “two-hit” combination of a carbocyanine compound of the present approach, and a second metabolic inhibitor (glycolysis or OXPHOS) to further starve the residual CSC population. The carbocyanine compound is used as a first metabolic inhibitor (specifically, as a mitochondria impairing agent) that serves as first-hit, followed by the use of a second metabolic inhibitor (for instance a glycolysis or an OXPHOS inhibitor) that acts as a second-hit.

Despite the acquisition of this compensatory glycolytic behavior, the carbocyanine compound treatment weakens CSCs by rendering the CSCs more sensitive to the action of glycolytic inhibitors and OXPHOS inhibitors. Thus, the effects of the carbocyanine compound allow for a variety of combination therapies. Under the present approach, a carbocyanine compound may be administered with one or more of such inhibitors, providing a “two-hit” therapeutic approach to eradicating CSCs. Demonstrative examples of the second metabolic inhibitor include glycolysis inhibitors Vitamin C and 2-deoxy-D-glucose (2-DG), as well as the OXPHOS inhibitors Doxycycline, Azithromycin, Niclosamide, and Berberine Chloride. Other FDA-approved members of the tetracycline family, including, for example, Tetracycline, Chlortetracycline, Minocycline, and Tigecycline, or the erythromycin family, including, for example, Erythromycin, Telithromycin, Clarithromycin, and Roxithromycin, may be used without departing form the present approach, Eravacycline, Sarecycline, and Omadacycline.

With respect to the active compounds, the demonstrative second inhibitor compounds are available in various forms in the art. For carbocyanine compounds such as, e.g., MDR, Cy5, and analogs thereof, the compound can be administered orally as a solid or as a liquid. In some embodiments, the carbocyanine compound can be administered intramuscularly, intravenously, or by inhalation as a solution, suspension, or emulsion. In some embodiments, the carbocyanine compound (which, for the avoidance of doubt, includes salts thereof) can be administered by inhalation, intravenously, or intramuscularly as a liposomal suspension. When administered through inhalation the active compound or salt can be in the form of a plurality of solid particles or droplets having any desired particle size, and for example, from about 0.001, 0.01, 0.1, or 0.5 microns, to about 5, 10, 20 or more microns, and optionally from about 1 to about 2 microns. It should be appreciated that the particular form of administration may vary, and that parameters outside of the scope of this disclosure (e.g., manufacturing, transportation, storage, shelf life, etc.) may be determinative of the common forms and concentrations of the carbocyanine compound.

Pharmaceutical compositions of the present approach include a carbocyanine compound (including salts thereof) as an active compound, in any pharmaceutically acceptable carrier. If a solution is desired, water may be the carrier of choice for water-soluble compounds or salts. With respect to water solubility, organic vehicles, such as glycerol, propylene glycol, polyethylene glycol, or mixtures thereof, can be suitable. Additionally, methods of increasing water solubility may be used without departing from the present approach. In the latter instance, the organic vehicle can contain a substantial amount of water. The solution in either instance can then be sterilized in a suitable manner known to those in the art, and for illustration by filtration through a 0.22-micron filter. Subsequent to sterilization, the solution can be dispensed into appropriate receptacles, such as depyrogenated glass vials. The dispensing is optionally done by an aseptic method. Sterilized closures can then be placed on the vials and, if desired, the vial contents can be lyophilized. Embodiments including a second inhibitor compound, such as a glycolysis inhibitor or an OXPHOS inhibitor, may co-administer a form of the second inhibitor available in the art. The present approach is not intended to be limited to a particular form of administration, unless otherwise stated.

In addition to the active compound(s), pharmaceutical formulations of the present approach can contain other additives known in the art. For example, some embodiments may include pH-adjusting agents, such as acids (e.g., hydrochloric acid), and bases or buffers (e.g., sodium acetate, sodium borate, sodium citrate, sodium gluconate, sodium lactate, and sodium phosphate). Some embodiments may include antimicrobial preservatives, such as methylparaben, propylparaben, and benzyl alcohol. An antimicrobial preservative is often included when the formulation is placed in a vial designed for multi-dose use. The pharmaceutical formulations described herein can be lyophilized using techniques well known in the art.

In embodiments involving oral administration of an active compound, the pharmaceutical composition can take the form of capsules, tablets, pills, powders, solutions, suspensions, and the like. Tablets containing various excipients such as sodium citrate, calcium carbonate and calcium phosphate may be employed along with various disintegrants such as starch (e.g., potato or tapioca starch) and certain complex silicates, together with binding agents such as polyvinylpyrrolidone, sucrose, gelatin and acacia. Additionally, lubricating agents such as magnesium stearate, sodium lauryl sulfate, and talc may be included for tableting purposes. Solid compositions of a similar type may be employed as fillers in soft and hard-filled gelatin capsules. Materials in this connection also include lactose or milk sugar, as well as high molecular weight polyethylene glycols. When aqueous suspensions and/or elixirs are desired for oral administration, the compounds of the presently disclosed subject matter can be combined with various sweetening agents, flavoring agents, coloring agents, emulsifying agents and/or suspending agents, as well as such diluents as water, ethanol, propylene glycol, glycerin and various like combinations thereof. In embodiments having a carbocyanine compound with a second inhibitor compound, the second inhibitor compound may be administered in a separate form, without limitation to the form of the carbocyanine compound.

Additional embodiments provided herein include liposomal formulations of the active compounds disclosed herein. The technology for forming liposomal suspensions is well known in the art. When the compound is an aqueous-soluble salt, using conventional liposome technology, the same can be incorporated into lipid vesicles. In such an instance, due to the water solubility of the active compound, the active compound can be substantially entrained within the hydrophilic center or core of the liposomes. The lipid layer employed can be of any conventional composition and can either contain cholesterol or can be cholesterol-free. When the active compound of interest is water-insoluble, again employing conventional liposome formation technology, the salt can be substantially entrained within the hydrophobic lipid bilayer that forms the structure of the liposome. In either instance, the liposomes that are produced can be reduced in size, as through the use of standard sonication and homogenization techniques. The liposomal formulations comprising the active compounds disclosed herein can be lyophilized to produce a lyophilizate, which can be reconstituted with a pharmaceutically acceptable carrier, such as water, to regenerate a liposomal suspension.

With respect to pharmaceutical compositions, the pharmaceutically effective amount of a carbocyanine compound described herein will be determined by the health care practitioner, and will depend on the condition, size and age of the patient, as well as the route of delivery. In one non-limited embodiment, a dosage from about 0.1 to about 200 mg/kg has therapeutic efficacy, wherein the weight ratio is the weight of the active compound, including the cases where a salt is employed, to the weight of the subject. In some embodiments, the dosage can be the amount of active compound needed to provide a serum concentration of the active compound of up to between about 1 and 5, 10, 20, 30, or 40 μM. In some embodiments, a dosage from about 1 mg/kg to about 10, and in some embodiments about 10 mg/kg to about 50 mg/kg, can be employed for oral administration. Typically, a dosage from about 0.5 mg/kg to 5 mg/kg can be employed for intramuscular injection. In some embodiments, dosages can be from about 1 μmol/kg to about 50 μmol/kg, or, optionally, between about 22 μmol/kg and about 33 μmol/kg of the compound for intravenous or oral administration. An oral dosage form can include any appropriate amount of active material, including for example from 5 mg to, 50, 100, 200, or 500 mg per tablet or other solid dosage form.

The following paragraphs describe the materials and methods used in connection with the data and embodiments set forth herein. It should be appreciated that those having an ordinary level of skill in the art may use alternative materials and methods generally accepted in the art, without deviating from the present approach.

Cell lines: Human breast cancer cell lines (MCF7, MDA-MB-231 and MDA-MB-468) were obtained from the American Type Culture Collection (ATCC). MitoTracker Deep Red FM (cat. no. M22426), a carbocyanine-based dye, was purchased from ThermoFisher Scientific, Inc. Poly(2-hydroxyethyl methacrylate) [poly-HEMA] was obtained from Sigma-Aldrich, Inc.

3D-Mammosphere Formation Assay: A single cell suspension was prepared using enzymatic (1× Trypsin-EDTA, Sigma Aldrich, cat. #T3924), and manual disaggregation (25 gauge needle). Five thousand cells were plated with in mammosphere medium (DMEM-F12/B27/20 ng/ml EGF/PenStrep), under non-adherent conditions, in six wells plates coated with 2-hydroxyethylmethacrylate (poly-HEMA, Sigma, cat. #P3932). Cells were grown for 5 days and maintained in a humidified incubator at 37° C. at an atmospheric pressure in 5% (v/v) carbon dioxide/air. After 5 days, 3D spheroids with a diameter greater than 50 μm were counted using a microscope, fitted with a graticule eye-piece, and the percentage of cells which formed spheroids was calculated and normalized to one (1=100% MFE; mammosphere forming efficiency). Maminosphere assays were performed in triplicate and repeated three times independently.

Metabolic Flux Analysis: Extracellular acidification rates and oxygen consumption rates were analyzed using the Seahorse XFe96 analyzer (Agilent/Seahorse Bioscience, USA). Cells were maintained in DMEM supplemented with 10% FBS (fetal bovine serum), 2 mM GlutaMAX, and 1% Pen-Strep. Forty-thousand breast cancer cells were seeded per well, into XFe96-well cell culture plates, and incubated at 37° C. in a 5% CO₂ humidified atmosphere. After 24-48 hours, MCF7 cells were washed in pre-warmed XF assay media, as previously described. ECAR and OCR measurements were normalized for cell protein content, by the SRB colorimetric assay. Data sets were analyzed using XFe96 software and Excel software.

Statistical Significance: Bar graphs are shown as the average±SEM (standard error of the mean). A p-value of less than 0.05 was considered statistically significant and is indicated by asterisks: *p<0.05, **p<0.01, ***p<0.001 and ****p<0.0001.

The terminology used in the description of embodiments of the present approach is for the purpose of describing particular embodiments only and is not intended to be limiting. As used in the description and the appended claims, the singular forms “a,” “an” and “the” are intended to include the plural forms as well, unless the context clearly indicates otherwise. The present approach encompasses numerous alternatives, modifications, and equivalents as will become apparent from consideration of the following detailed description.

It will be understood that although the terms “first,” “second,” “third,” “a),” “b),” and “c),” etc. may be used herein to describe various elements of the present approach, and the claims should not be limited by these terms. These terms are only used to distinguish one element of the present approach from another. Thus, a first element discussed below could be termed an element aspect, and similarly, a third without departing from the teachings of the present approach. Thus, the terms “first,” “second,” “third,” “a),” “b),” and “c),” etc. are not intended to necessarily convey a sequence or other hierarchy to the associated elements but are used for identification purposes only. The sequence of operations (or steps) is not limited to the order presented in the claims.

Unless otherwise defined, all terms (including technical and scientific terms) used herein have the same meaning as commonly understood by one of ordinary skill in the art. It will be further understood that terms, such as those defined in commonly used dictionaries, should be interpreted as having a meaning that is consistent with their meaning in the context of the present application and relevant art and should not be interpreted in an idealized or overly formal sense unless expressly so defined herein. All publications, patent applications, patents and other references mentioned herein are incorporated by reference in their entirety. In case of a conflict in terminology, the present specification is controlling.

Also, as used herein, “and/or” refers to and encompasses any and all possible combinations of one or more of the associated listed items, as well as the lack of combinations when interpreted in the alternative (“or”).

Unless the context indicates otherwise, it is specifically intended that the various features of the present approach described herein can be used in any combination. Moreover, the present approach also contemplates that in some embodiments, any feature or combination of features described with respect to demonstrative embodiments can be excluded or omitted.

As used herein, the transitional phrase “consisting essentially of” (and grammatical variants) is to be interpreted as encompassing the recited materials or steps “and those that do not materially affect the basic and novel characteristic(s)” of the claim. Thus, the term “consisting essentially of” as used herein should not be interpreted as equivalent to “comprising.”

The term “about,” as used herein when referring to a measurable value, such as, for example, an amount or concentration and the like, is meant to encompass variations of ±20%, ±10%, ±5%, ±1%, ±0.5%, or even ±0.1% of the specified amount. A range provided herein for a measurable value may include any other range and/or individual value therein.

Having thus described certain embodiments of the present approach, it is to be understood that the scope of the appended claims is not to be limited by particular details set forth in the above description as many apparent variations thereof are possible without departing from the spirit or scope thereof as hereinafter claimed.

1. A method for treating cancer in a patient, wherein the method comprises administering a pharmaceutically effective amount of a carbocyanine compound to the patient.

2. The method of claim 1, wherein the carbocyanine compound comprises one of MitoTracker Deep Red (1-{4-[(chloromethyl)phenyl]methyl}-3,3-dimethyl-2-[5-(1,3,3-trimethyl-1,3-dihydro-2H-indol-2-ylidene)penta-1,3-dien-1-yl]-3H-indolium chloride), HITC Iodide (B-1,1′,3,3,3′,3′-Hexamethylindotricarbocyanine iodide), and DDI, (1,1′Diethyl-2-2′-dicarboccyanine iodide).

3. The method of claim 1, wherein the carbocyanine compound comprises a compound having the chemical structure 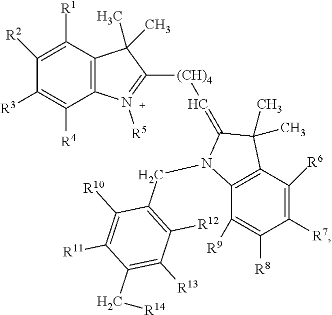
wherein each of R¹ through R¹⁴ may be the same or different, and is selected from hydrogen, carbon, nitrogen, sulfur, oxygen, fluorine, chlorine, bromine, iodine, carboxyl, alkanes, cyclic alkanes, alkane-based derivatives, alkenes, cyclic alkenes, alkene-based derivatives, alkynes, alkyne-based derivative, ketones, ketone-based derivatives, aldehydes, aldehyde-based derivatives, carboxylic acids, carboxylic acid-based derivatives, ethers, ether-based derivatives, esters and ester-based derivatives, amines, amino-based derivatives, amides, amide-based derivatives, monocyclic or polycyclic arene, heteroarenes, arene-based derivatives, heteroarene-based derivatives, phenols, phenol-based derivatives, benzoic acid, benzoic acid-based derivatives, membrane-targeting signals, and mitochondria-targeting signals, provided that at least one of R¹ through R¹⁴ is not H.

4. The method of claim 1, wherein the carbocyanine compound comprises a compound having the chemical structure 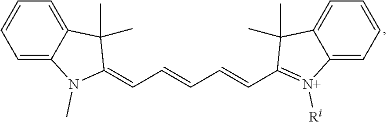
wherein Rⁱ is selected from

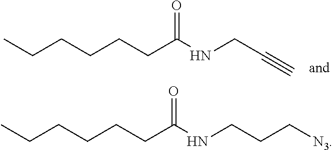

5. The method of claim 1, further comprising administering to the patient a second inhibitor compound selected from one of a glycolysis inhibitor compound and an OXPHOS inhibitor compound.

6. The method of claim 5, wherein the second inhibitor compound comprises one of Vitamin C, 2-deoxy-glucose, Doxycycline, Niclosamide, and Berberine chloride.

7. The method of claim 1, wherein the method comprises one of inhibiting mitochondrial metabolism in the cancer, eradicating cancer stem cells (CSCs) in the cancer, inhibiting propagation of the cancer, preventing metastasis, and preventing recurrence.

8. A pharmaceutical composition comprising a carbocyanine compound.

9. The pharmaceutical composition of claim 8, wherein the carbocyanine compound comprises one of MitoTracker Deep Red (1-{4-[(chloromethyl)phenyl]methyl}-3,3-dimethyl-2-[5-(1,3,3-trimethyl-1,3-dihydro-2H-indol-2-ylidene)penta-1,3-dien-1-yl]-3H-indolium chloride), HITC Iodide (B-1,1′,3,3,3′,3′-Hexamethylindotricarbocyanine iodide), and DDI, (1,1′Diethyl-2-2′-dicarboccyanine iodide).

10. The pharmaceutical composition of claim 8, wherein the carbocyanine compound comprises a compound having the chemical structure 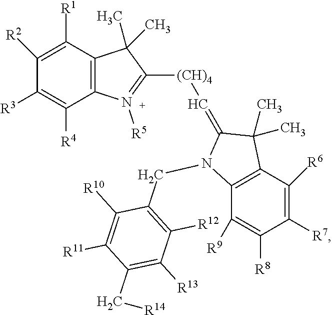
wherein each of R¹ through R¹⁴ may be the same or different, and is selected from hydrogen, carbon, nitrogen, sulfur, oxygen, fluorine, chlorine, bromine, iodine, carboxyl, alkanes, cyclic alkanes, alkane-based derivatives, alkenes, cyclic alkenes, alkene-based derivatives, alkynes, alkyne-based derivative, ketones, ketone-based derivatives, aldehydes, aldehyde-based derivatives, carboxylic acids, carboxylic acid-based derivatives, ethers, ether-based derivatives, esters and ester-based derivatives, amines, amino-based derivatives, amides, amide-based derivatives, monocyclic or polycyclic arene, heteroarenes, arene-based derivatives, heteroarene-based derivatives, phenols, phenol-based derivatives, benzoic acid, benzoic acid-based derivatives, membrane-targeting signals, and mitochondria-targeting signals, provided that at least one of R¹ through R¹⁴ is not H.

11. The pharmaceutical composition of claim 8, wherein the carbocyanine compound comprises a compound having the chemical structure 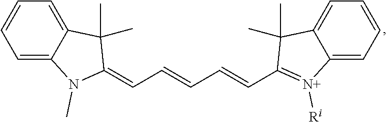
wherein Rⁱ is selected from

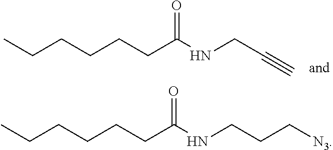

12. The pharmaceutical composition of claim 8, further a second inhibitor compound selected from one of a glycolysis inhibitor compound and an OXPHOS inhibitor compound.

13. The pharmaceutical composition of claim 12, wherein the second inhibitor compound comprises one of Vitamin C, 2-deoxy-glucose, Doxycycline, Niclosamide, and Berberine chloride.

14. The pharmaceutical composition of claim 10, wherein each of R¹ through R⁴ and R⁶ through R¹³ is hydrogen, R⁵ is methyl, and R¹⁴ is chlorine.

15. The pharmaceutical composition of claim 10, wherein at least one of R¹ through R¹⁴ is a membrane-targeting signal.

16. The pharmaceutical composition of claim 15, wherein the membrane-targeting signal comprises one of palmitic acid, stearic acid, myristic acid, oleic acid, a short-chain fatty acid, and a medium-chain fatty acid.

17. The pharmaceutical composition of claim 10, wherein at least one of R¹ through R¹⁴ is a mitochondrial-targeting signal.

18. The pharmaceutical composition of claim 17, wherein the mitochondrial-targeting signal is one of tri-phenyl-phosphonium (TPP), a TPP-derivative, a lipophilic cation, and 10-N-nonyl acridine orange.

19. The use of a carbocyanine compound in the manufacture of a medicament for treating cancer.

20. The use of claim 19, wherein the carbocyanine compound comprises one of MitoTracker Deep Red (1-{4-[(chloromethyl)phenyl]methyl}-3,3-dimethyl-2-[5-(1,3,3-trimethyl-1,3-dihydro-2H-indol-2-ylidene)penta-1,3-dien-1-yl]-3H-indolium chloride), HITC Iodide (B-1,1′,3,3,3′,3′-Hexamethylindotricarbocyanine iodide), and DDI, (1,1′Diethyl-2-2′-dicarboccyanine iodide).

21. The use of claim 19, wherein the carbocyanine compound comprises one of a compound having the chemical structure 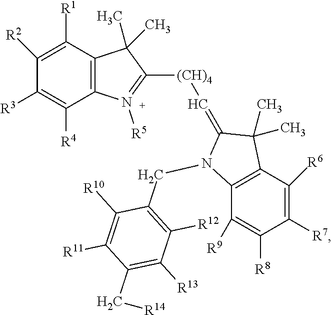
wherein each of R¹ through R¹⁴ may be the same or different, and is selected from hydrogen, carbon, nitrogen, sulfur, oxygen, fluorine, chlorine, bromine, iodine, carboxyl, alkanes, cyclic alkanes, alkane-based derivatives, alkenes, cyclic alkenes, alkene-based derivatives, alkynes, alkyne-based derivative, ketones, ketone-based derivatives, aldehydes, aldehyde-based derivatives, carboxylic acids, carboxylic acid-based derivatives, ethers, ether-based derivatives, esters and ester-based derivatives, amines, amino-based derivatives, amides, amide-based derivatives, monocyclic or polycyclic arene, heteroarenes, arene-based derivatives, heteroarene-based derivatives, phenols, phenol-based derivatives, benzoic acid, benzoic acid-based derivatives, membrane-targeting signals, and mitochondria-targeting signals, provided that at least one of R¹ through R¹⁴ is not H.

22. The use of claim 19, wherein the carbocyanine compound comprises a compound having the chemical structure 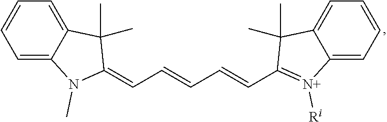
wherein Rⁱ is selected from

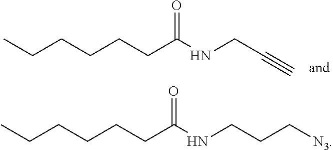

23. The use of claim 19, wherein the medicament treats cancer through at least one of inhibiting mitochondrial metabolism in the cancer, eradicating cancer stem cells (CSCs) in the cancer, inhibiting propagation of the cancer, preventing metastasis, and preventing recurrence.
